(19) United States
(12) Patent Application Publication
(10) Pub. No.: US 2025/0072911 A1
(43) Pub. Date: Mar. 6, 2025

(54) TRANSHUMERAL GLENOID TECHNIQUES AND INSTRUMENTATION FOR USE IN TISSUE SPARING SHOULDER ARTHROPLASTIES

(71) Applicant: DePuy Synthes Products, Inc., Raynham, MA (US)

(72) Inventors: Stephen J. Orphanos, Bridgewater, MA (US); Mollie Waters, Boston, MA (US); Jae S. Stelzer, Raynham, MA (US); Paul Favorito, Cincinnati, OH (US); Peter Chalmers, Salt Lake City, UT (US); Robert Ciocca, Woonsocket, RI (US); Anders Ekelund, Bromma (SE)

(21) Appl. No.: 18/823,275

(22) Filed: Sep. 3, 2024

Related U.S. Application Data
(60) Provisional application No. 63/579,947, filed on Aug. 31, 2023.

(51) Int. Cl. A61B 17/16; A61F 2/40

(52) U.S. Cl. CPC A61B 17/1684; A61B 17/1624; A61F 2/4081

(57) ABSTRACT

Systems, devices, and methods for preparing a glenoid to receive an implant using a transhumeral approach are disclosed. Various tools, such as a reamer attachment, can be disposed in a glenohumeral space proximate to the glenoid, and then a driver or other shaft can be separately introduced into the glenohumeral space. The driver can be coupled to the reamer attachment and then operated to treat or otherwise prepare the glenoid to receive an implant. After the reamer attachment is no longer needed to perform the desired treatment, it can be detached from the driver and both the reamer attachment and driver can be removed from the glenohumeral space. The disclosed systems, devices, and methods can be performed with or without a guidewire, and various other instruments, such as those for identifying a center of the glenoid, can be used.

### CROSS REFERENCE TO RELATED APPLICATION

The present disclosure claims priority to and the benefit of U.S. Provisional Patent Application No. 63/579,947, entitled “Transhumeral Glenoid Techniques and Instrumentation for Use in Tissue Sparing Shoulder Arthroplasties,” filed Aug. 31, 2023, the disclosure of which is incorporated by reference herein in its entirety.

### FIELD

The present disclosure relates to devices, methods, and techniques used in shoulder repair procedures, such as shoulder arthroplastics, and more particularly relates to devices and associated methods used to prepare a glenoid in a human shoulder to receive an implant and/or a prosthetic while allowing surrounding tendons such as the subscapularis tendon to remain intact, the methods involving a transhumeral approach.

### BACKGROUND

During the lifetime of a patient, it may be necessary to perform a shoulder arthroplasty due to, for example, disease or trauma. Various forms of this type of surgery exist, with the overarching purpose being to remove and/or replace damaged or missing portions of the anatomy with prosthetic components. For example, as shown in FIG. 1A, in an anatomic total shoulder arthroplasty, a humeral prosthesis 10 can be used to replace the natural head of a patient's humerus. The humeral prosthesis 10 typically includes an elongated post component 12 that is implanted into an intramedullary canal of the patient's humerus and a hemispherical-shaped prosthetic head component 14 that is secured to the post component 12. Additionally, the natural glenoid surface of the scapula can be resurfaced or otherwise replaced with an anatomic glenoid implant 20. An anatomic glenoid implant 20 typically includes a concave bearing surface 24 upon which the prosthetic head component 14 of the humeral prosthesis 10 articulates. A peg or keel 22 can project from the distal end of the implant 20 and can be secured (e.g., cemented) into the glenoid cavity of the patient's scapula.

While FIG. 1A provides for an anatomic total shoulder arthroplasty, in other instances, the arthroplasty may be a partial shoulder arthroplasty, meaning only a portion of the shoulder anatomy may be replaced with an implant. This may include, for example, only providing for a humeral prosthesis without a glenoid implant, or a glenoid implant without a humeral prosthesis, among other variations appreciated by those skilled in the art. Further, while FIG. 1A provides for an anatomic procedure, reverse procedures are also known procedures for repairing shoulders. They can be helpful, for example, when a patient's natural shoulder has degenerated to a severe degree of joint instability and pain. In a reverse procedure, the mechanics of the shoulder can be changed, reversing the anatomy, or structure, of the healthy shoulder. For example, as shown in FIG. 1B, in a reverse total shoulder arthroplasty, a humeral prosthesis 50 can be used to replace the natural head of the patient's humerus. The humeral prosthesis 50 typically includes an elongated post component 52 that is implanted into an intramedullary canal of the patient's humerus and a concave-shaped prosthetic head component 54, known as a humeral cup, is secured to the post component 52. Additionally, a reverse glenoid implant, e.g., a hemispherical-shaped glenosphere 60, can be secured to the glenoid bone of the patient's scapula. Such a reverse configuration allows the patient's deltoid muscle, which is one of the larger and stronger shoulder muscles, to raise the arm.

During various types of shoulder arthroplasty surgeries, there is soft tissue impeding access to the surgical arthroplasty site. This tissue typically includes a patient's subscapularis tendon. In many traditional surgical approaches, the subscapularis tendon is detached from a humeral attachment point on the humerus to provide better access to the surgical site. In at least some arthroplasty procedures, the humerus is subsequently externally rotated to allow access to the joint space, essentially dislocating the humeral head. This provides a surgeon full visibility to the humeral head, as well as the glenoid after humeral head resection. In an anatomical procedure in which the humeral head is being replaced, once the humeral head is exposed, the convex portion of the bone is resected to a flat plane (referred to herein as a “humeral resection surface”) and prepared to receive a humeral implant and/or prosthesis. The glenoid is fully visible, thus allowing it to be accessed and prepared to receive the humeral implant and/or prosthesis. This can include placing an implant and/or prosthesis at the glenoid surface to receive the humeral implant and/or prosthesis and/or preparing the surface of the glenoid in a manner that it can receive the humeral implant and/or prosthesis. In a reverse procedure, the location where the humeral head is typically located can be prepared to receive a prosthetic head component (e.g., a humeral cup) after the proximal portion of the humerus is externally rotated, while the glenoid can be prepared to receive the humeral implant and/or prosthesis (e.g., a hemispherical-shaped glenosphere).

Upon gaining sufficient access to the surgical site, surgeons often employ various tools to create the proper geometry within the glenoid surface. This geometry depends, at least in part, on the chosen implant and/or prosthesis and/or any anatomical or disease issues associated with the patient at the surgical site to ensure that the implant and/or prosthesis fits securely with respect to the bone so it can perform properly within the joint space. Generally to create such a geometry, the glenoid surface is reamed, cut, or otherwise shaped to the desired shape prior to inserting an implant and/or prosthesis. Existing reaming tools, among other instruments used in such procedures, are designed for use in procedures in which the subscapularis tendon has been detached from its humeral attachment point to allow a full 360° view of the glenoid anatomy for treatment of the same. The size, shape, and function of such tools are based on having access and visualization to the joint space that is not inhibited by the subscapularis tendon and/or other tissue that may impede access to the surgical site. Access to a deeper anatomy, such as the glenoid, is aided by the lack of inhibition provide by the subscapularis tendon and/or other tissue. Following surgery, the subscapularis tendon is reattached. Repair and healing of the tendon are critical to the proper function of the joint and incomplete healing results in complications, pain, and instability.

Alternatively, the procedure can be performed leaving the subscapularis tendon attached-referred to as tissue sparing-such that the surgeon(s) works only within the limited joint space superior and inferior to the tissue borders of the subscapularis. In such instances, because the surgeon is not able to externally rotate and essentially dislocate the humeral head, he or she has limited access to a deeper anatomy like the surface of the glenoid. It can be difficult to make assessments of the surgical site and/or otherwise provide treatment to a surface like that of the glenoid. To the extent the surgeon uses existing arthroplasty tools that are not specifically designed for a tissue sparing approach, he or she manipulates the subscapularis tendon during tool insertion to allow the tools to access the surgical site. This is because traditional tools are not suitable and/or designed for use in such a tissue sparing procedure. Beyond the visual complications noted above, the narrow joint space makes it difficult for the surgeon to apply the requisite force needed to perform tasks like reaming, and provide such force in a consistent manner, across the glenoid surface. Further, traditional arthroplasty tools are not being used during transhumeral procedures at least because, prior to the present disclosure, transhumeral approaches for shoulder arthroplasty were not being performed. At least because existing arthroplasty tools are designed for use when the subscapularis tendon has been detached, they are too bulky and cumbersome to use in a tissue sparing procedure. Existing tools do not have the versatility in movement, size, and function to access a limited joint space and provide consistent and sufficient force to adequately prepare a glenoid surface to receive an implant and/or prosthesis. They are not designed to be low profile so as to be able to work within confined spaces, nor are they modular to allow for assembly/disassembly within the joint space. Further, to the extent existing devices have at least some such capabilities, they are not typically easy to use, meaning existing devices are insufficient to provide suitable usability to surgeons and the like.

Accordingly, there is a need for glenoid arthroplasty tools and instruments, and related methods, for use in tissue sparing arthroplasty procedures where access is limited while also minimizing damage to surrounding soft tissue, and adjacent neurovascular and boney structures.

### SUMMARY

The present disclosure is generally directed to devices, systems, and methods that allow for the preparation of a glenoid in conjunction with shoulder arthroplasties or other types of surgical procedures to be done with a transhumeral approach. More specifically, as provided for herein, the primary or main bone preparation steps are performed with a transhumeral approach, but that does not mean that each and every step of an entire procedure must be performed transhumerally. Certain portions of one or more procedures may be performed with a non-transhumeral approach, such as within a rotator interval while the subscapularis is intact but performed non-transhumerally. Throughout the present disclosure, references to certain aspects of a procedure involving a transhumeral approach encompasses these variations, and more generally refers to the primary bone preparation steps, particularly with respect to the glenoid, being performed with a transhumeral approach.

The tools to be used in conjunction with treating or otherwise preparing a surface of the glenoid to receive an implant and/or prosthesis can be introduced separate from components that are used to operate the tools, such as drivers or other shafts. For example, a reamer attachment can be introduced to a glenohumeral joint space adjacent to the glenoid being treated through a rotator interval and then a driver configured to operate the reamer attachment can be introduced into the glenohumeral joint space through a transhumeral bone tunnel formed in the humerus. The reamer attachment and driver can be coupled together within the glenohumeral joint space, with the driver then being operated to rotate and translate the reamer attachment so that it reams the surface of the glenoid in preparation for receiving an implant (e.g., a prosthesis). Once the reamer attachment performs the desired tasks, it can be decoupled from the driver and evacuated from the surgical site. Likewise, the driver can be evacuated from the surgical site, or alternatively, it can remain to receive other tools for treatment of the glenoid. Notably, the tools and related disclosures herein enable the ability to perform tasks, such as reaming a glenoid, while the subscapularis tendon remains intact with a humeral attachment point for the duration of the surgical procedure. Further, the present disclosure accounts for multiple types of transhumeral humeral approaches, including approaches when the arm is in adduction and when it is not in adduction.

Prior to performing actions like reaming the surface of the glenoid, it can be appropriate to locate and/or otherwise denote a center or approximate center of a surface of the glenoid. The present disclosure provides for devices, systems, and methods for taking these actions as well. This can include employing a guide that mates to the humerus and helps set a path at which the aforementioned transhumeral bone tunnel is to be formed. The resulting transhumeral bone tunnel can then allow access to the center or approximate center of the glenoid. The guide can be designed specifically for use with the glenoid, as opposed to having a dual purpose of also being used in conjunction with performing similar types of actions (e.g., reaming), on a humerus. The guide can be mated to the humerus, for example using a drill cannula. Subsequently, a drill or drill bit can be passed through the drill cannula, through the humerus to form the transhumeral bone tunnel, and then into a surface of the glenoid to form a starter and/or pilot hole. Upon identification of the center or approximate center of the glenoid, the surface of the glenoid can be treated to prepare it to receive an implant(s), such as by reaming the glenoid, as referenced above. Identifying and denoting the center, approximate center, or another location, can be achieved, for example, by employing one or more glenoid sizer or sizer plates. The glenoid sizer(s) can also be used to help determine the desired size of an implant(s) to be disposed in the glenoid and/or desired actions to be performed on the glenoid (e.g., reaming), and/or resulting configurations of the glenoid surface in view of the same.

In alternative embodiments, a guide, also referred to as a humeral guide, that can be used for guiding procedures to be performed both at the glenoid and the humerus can be used to provide transhumeral treatment to the glenoid. In such embodiments, the guide can help set a path at which a transhumeral bone tunnel can be formed, with the bone tunnel allowing access to a center or approximate center, or another location, of a humeral resection surface, as well as a center or approximate center, or another location, of the glenoid. The guide can include a sizer attachment coupled to a distal end of an arm of the guide, the sizer attachment being designed to help locate and demarcate the center or approximate center with respect to one or both bones (i.e., the humerus and glenoid), as well as determine appropriate sizes for instrumentation and/or implants to be used in conjunction with the procedure. For example, various sizer plates can be used in conjunction with the same. The guide can be mated to the humerus, for example using a drill cannula and/or using the sizer attachment. Subsequently, a drill or drill bit can be passed through the cannulated drill guide and through the humerus to form the transhumeral bone tunnel. Upon existing the humerus, the drill bit can pass through the sizer attachment, and, eventually, into a surface of the glenoid to form a starter and/or pilot hole.

Regardless of whether a guide designed principally for use in identifying a center or approximate center, or another location, of a glenoid is used or a guide designed for use in identifying centers or approximate centers, or other locations, of both a humeral resection surface and a glenoid is used, the actions of locating and demarcating the center or approximate center, or another location, of the glenoid can be performed similarly. More particularly, a drill can be passed into and through the drill cannula to allow a starter hole or pilot hole to be formed in the surface of the glenoid. Subsequently, the drill can be backed out and a driver can be introduced to perform reaming of the glenoid. Depending on whether a guidewire is desired for use of tools like the reamer attachment, the drill can be disposed in the pilot hole surface of the glenoid to serve as a guidewire or the drill can be fully removed and a guidewire can be placed in the pilot hole formed by the drill.

One embodiment a method for reaming a glenoid includes disposing a reamer attachment in a glenohumeral joint space, with the glenohumeral joint space being located between a humerus and a glenoid. The method further includes passing a driver through a transhumeral tunnel formed in the humerus such that a distal end of the driver extends into the glenohumeral joint space, coupling the reamer attachment to the distal end of the driver, and operating the driver to rotate the reamer attachment and advance the reamer attachment into a surface of the glenoid.

In at least some embodiments, prior to disposing the reamer attachment in the glenohumeral joint space, the method further includes coupling a glenoid guide to the humerus such that a drill cannula associated with the glenoid guide is in contact with the humerus and passing a drill through the drill cannula to form the transhumeral tunnel. In at least some such embodiments, the method can further include operating a drill to form a pilot hole in a surface of the glenoid, docking a tip of the drill in the pilot hole, and adjusting a location of the glenoid guide with respect to the humerus to set at least one of a version or inclination for the glenoid guide. The method can also include disposing a glenoid sizer having an open slot formed in the sizer in the glenohumeral joint space and passing the glenoid sizer onto the drill, with the drill passing through the slot and to a central opening of the glenoid sizer. Such methods can further include using the glenoid sizer to at least one of identify an approximate center of the surface of the glenoid, determine a size of the glenoid, and/or identify a trajectory for using the glenoid guide to ream the surface of the glenoid.

The glenoid guide can include, for example, an offset arm that can have a proximal portion, a distal portion, and an intermediate portion. The offset arm can further include a proximal opening formed in the proximal portion and a distal opening formed in the distal portion, with the proximal and distal openings being colinear. The intermediate portion can extend offset from a longitudinal axis that extends between the proximal and distal openings. In such embodiments, the longitudinal axis can extend through a humerus when coupled to the humerus while the intermediate portion can extend around the humerus.

In at least some embodiments, prior to disposing the reamer attachment in the glenohumeral joint space, the method can include coupling a humeral guide to the humerus such that a drill cannula associated with the humeral guide is in contact with the humerus and a humeral sizer attachment coupled to the humeral guide is in contact with a humeral resection surface. Such methods can further include passing a drill through the drill cannula to form the transhumeral tunnel. In at least some such embodiments, the method can further include locating an approximate center of the humeral resection surface, with the approximate center being located at a terminal end of the transhumeral tunnel. The action of coupling a humeral guide to the humerus can include inserting at least one bone pin into the humerus. The bone pin can be coupled to a bone pin clamp, which can be coupled to the humeral guide. In at least some such embodiments, the method can further include adjusting the bone pin clamp in at least one degree of freedom, and/or in at least two degrees of freedom, to position the at least one bone pin at a desired location to be inserted into the humerus.

The action of locating an approximate center of a humeral resection surface can include coupling a proximal end of the humeral sizer attachment to a distal end of an arm of the humeral guide and positioning a distal end of the humeral sizer attachment at a location proximate to the humeral resection surface. The distal end of the sizer attachment can include a plate having a central opening formed therein. The method can also include positioning the central opening of the plate of the distal end of the humeral sizer attachment such that it aligns with a longitudinal axis extending through the drill cannula and the transhumeral tunnel. In at least some such embodiments, the drill cannula can be moveably coupled to a proximal end of the arm of the humeral guide.

Prior to disposing the reamer attachment in the glenohumeral joint space, the method can include disposing a glenoid sizer in the glenohumeral joint space and passing a shaft through the transhumeral tunnel formed in the humerus such that a distal end of the shaft extends into the glenohumeral joint space. The method can further include coupling the glenoid sizer to the distal end of the shaft and operating the shaft to cause the glenoid sizer to be proximate to and/or in contact with the surface of the glenoid in conjunction with at least one of identifying an approximate center of the surface of the glenoid or determining a size of the glenoid. The action of disposing a glenoid sizer in the glenohumeral joint space can include passing a sizer handle having the glenoid sizer coupled to a distal end of the sizer handle through a rotator interval to dispose the glenoid sizer in the glenohumeral joint space.

Prior to disposing the reamer attachment in the glenohumeral joint space, the method can include passing a drill bit into and through the glenohumeral joint space and into a surface of the glenoid to form at least one of a starter hole or a pilot hole, with the drill bit not passing through the humerus. In at least some embodiments, prior to disposing the reamer attachment in the glenohumeral joint space, the method can include passing a drill bit through the transhumeral tunnel formed in the humerus, through the glenohumeral joint space, and into a surface of the glenoid to form at least one of a starter hole or a pilot hole. The at least one of a starter hole or a pilot hole in any instances provided can be formed at an identified approximate center of the surface of the glenoid. In at least some embodiments, the drill bit can be a stepped drill bit having a distal portion with a smaller diameter than a portion of the drill bit that is proximal of the distal portion.

At least some of the embodiments provided for herein can utilize a guidewire in conjunction with the method. For example, the method can further include disposing a guidewire in the surface of the glenoid and disposing the driver over the guidewire. Operating the driver to rotate the reamer attachment and advance the reamer attachment into a surface of the glenoid can occur while the guidewire is disposed within the driver and the reamer attachment can be disposed radially around the guidewire.

In at least some embodiments, the method can include engaging a distal tip of the driver with a pilot hole formed in an approximate center of the surface of the glenoid prior to operating the driver to rotate the reamer attachment and advance the reamer attachment into the surface of the glenoid. In at least some such embodiments, the method can be performed without the assistance of a guidewire disposed in the surface of the glenoid.

The method can also include inserting an implant into the surface of the glenoid after the reamer attachment has treated the surface of the glenoid. This can occur, for example, by decoupling the reamer attachment from the driver, removing the reamer attachment and the driver from the glenohumeral joint space, passing a distal end of the glenoid guide into the glenohumeral joint space, with the distal end of the glenoid guide having coupled to it, the implant, and applying a force to a proximal end of the glenoid guide to dispose the implant in the surface of the glenoid. In at least some such embodiments, the method can further include decoupling the implant from distal end of the glenoid guide, removing the glenoid guide from the glenohumeral joint space, passing the distal end of the glenoid guide into the glenohumeral joint space, with the distal end of the glenoid guide having coupled to it an impactor, and applying a force to the proximal end of the glenoid guide to use the impactor to further dispose the implant in the surface of the glenoid.

Prior to inserting an implant into the surface of the glenoid and after the reamer attachment has treated the surface of the glenoid, the method can include passing a distal end of the glenoid guide into the glenohumeral joint space, with the distal end of the glenoid guide having coupled thereto an implant trial. The method can further include applying a force to a proximal end of the glenoid guide to dispose the implant trial in the surface of the glenoid and using the implant trial to determine at least one of an appropriate configuration of the implant, a size of the implant, or a location to dispose the implant in the surface of the glenoid.

For the actions of using the implant trial, the glenoid guide can include, for example, an offset arm that can have a proximal portion, a distal portion, and an intermediate portion. The offset arm can further include a proximal opening formed in the proximal portion and a distal opening formed in the distal portion, with the proximal and distal openings being colinear. The intermediate portion can extend offset from a longitudinal axis that extends between the proximal and distal openings. In such embodiments, the longitudinal axis can extend through a humerus when coupled to the humerus while the intermediate portion can extend around the humerus.

All of the methods provided for above can be performed while having a subscapularis tendon intact during an entirety of the method. In at least some embodiments, the reamer attachment can be inserted to the glenohumeral joint space at least one of superior to the intact subscapularis tendon or inferior to the intact subscapularis tendon. The method can further include manipulating the subscapularis tendon to increase visibility by moving it away from its natural location while keeping it intact.

All of the methods provided for above can be performed while the humerus is not distracted from its associated joint. Further, all of the methods provided for herein can be performed while the humerus is in an adducted position.

One embodiment of a system for reaming a glenoid includes a reamer attachment and a driver. The reamer attachment includes a central opening formed through it and one or more cutting surfaces disposed radially outward from the central opening. The driver is configured to pass into the central opening of the reamer attachment to selectively couple and de-couple from the reamer attachment within a glenohumeral joint space. As a result, the reamer attachment and the driver can be disposed within the glenohumeral joint space separate from each other.

In at least some embodiments, the one or more cutting surfaces disposed radially outward from the central opening include a first cutting surface disposed at a distal end of the reamer attachment and a second cutting surface disposed proximal of the first cutting surface, with the second cutting surface being disposed further radially outward from the central opening than the first cutting surface. An inner wall of the reamer attachment can include a keyed portion. Further, the driver can also include a keyed portion that is complementary to the keyed portion of the reamer attachment such that when the keyed portion of the reamer attachment is mated with the keyed portion of the driver, the reamer attachment and driver can be coupled together in a manner that allows the driver to rotate and translate the reamer attachment.

A distal tip of the driver can include a bullet-shaped tip. The system can also include a glenoid guide. The glenoid guide can have an offset arm that has a proximal portion, a distal portion, and an intermediate portion. The offset arm can further include a proximal opening formed in the proximal portion and a distal opening formed in the distal portion, with the proximal and distal openings being colinear. The intermediate portion can extend offset from a longitudinal axis that extends between the proximal and distal openings. In such embodiments, the longitudinal axis can extend through a humerus when coupled to the humerus while the intermediate portion can extend around the humerus. In at least some embodiments, the glenoid guide can include a drill cannula. The drill cannula can be configured to be coupled to the proximal portion of the arm. Further, the drill cannula can be able to pass through and be selectively held within the proximal opening by the glenoid guide. The glenoid guide can also include a post disposed as part of the distal portion. The post can be configured to receive an implant on it.

In at least some embodiments, the system can include a glenoid sizer having a central opening formed in the sizer. The glenoid sizer can be configured to mate to at least one of the driver or another shaft. Further, the glenoid sizer can be configured to assist in identifying an approximate center of the glenoid and/or determining a size or one or more implants to be used in conjunction with the glenoid.

One embodiment of a glenoid guide includes a proximal portion, a distal portion, and an offset arm extending from the proximal portion to the distal portion. The proximal portion includes a proximal opening formed in it, and the distal portion includes a distal opening formed in it. The distal and proximal openings are colinear. The offset arm is offset with respect to a longitudinal axis extending through the distal and proximal openings. The glenoid guide is configured such that the longitudinal axis can extend through a humerus when coupled to the humerus while the offset arm extends around the humerus.

The glenoid guide can also include a drill cannula. The drill cannula can be configured to be coupled to the proximal portion. Further, the drill cannula can be able to pass through and be selectively held within the proximal opening by the glenoid guide. In at least some embodiments, the glenoid guide can also include a post disposed as part of the distal portion. The post can be configured to receive an implant on it.

Any of the features or variations described herein can be applied to any particular aspect or embodiment of the present disclosure in a number of different combinations. The absence of explicit recitation of any particular combination is due solely to avoiding unnecessary length or repetition.

### BRIEF DESCRIPTION OF DRAWINGS

This disclosure will be more fully understood from the following detailed description taken in conjunction with the accompanying drawings, in which:

FIG. 1A is a side, partially translucent view of one example of an anatomic shoulder joint reconstruction, including an anatomic glenoid implant of the prior art coupled to a scapula;

FIG. 1B is a side, partially translucent view of one example of reverse shoulder joint reconstruction, including a reverse glenoid implant of the prior art coupled to a scapula;

FIG. 2A is a perspective view of a human glenohumeral shoulder joint, and associated joint space, including a humerus having a resected humeral head and an elongate shaft;

FIG. 2B is a perspective view of the human glenohumeral shoulder joint of FIG. 2A with the humerus aligned in position for glenoid preparation;

FIG. 3 is a side perspective view of one embodiment of a glenoid guide that includes a rigid arm, a drill cannula, and a drill bit disposed within the drill cannula;

FIG. 4A is a perspective view of the human glenohumeral shoulder joint of FIG. 2A, the glenoid guide of FIG. 3 coupled to the humeral resection surface, and the drill bit extending through a transhumeral tunnel formed in the humerus and exiting through the humeral resection surface and into a glenoid surface;

FIG. 4B is a side view of the drill bit of FIG. 4A;

FIG. 5A is a side view of an embodiment of a reamer attachment coupled to a driver within joint space of the human glenohumeral shoulder joint of FIG. 4A;

FIG. 5B is a bottom perspective view of the reamer attachment of FIG. 5A;

FIG. 5C is a side, cross-sectional view of the reamer attachment of FIG. 5B taken along line C-C;

FIG. 5D is a side perspective view of a distal end of the driver of FIG. 5A;

FIG. 5E is a side, fully cross-sectional view of the reamer attachment and the driver of FIG. 5A taken along line E-E;

FIG. 6A is a perspective view of the human glenohumeral shoulder joint of FIG. 4A including the glenoid surface, the humerus (with the humeral resection surface) in an adduction position, the figure also illustrating a sizer handle positioning a glenoid sizer proximate to the glenoid surface and the drill bit of FIG. 4B being passed through a transhumeral opening formed in the humerus to engage with the glenoid sizer and glenoid surface;

FIG. 6B is a detailed perspective view of the human glenohumeral shoulder joint and related anatomies, the sizer handle, the glenoid sizer, and the drill bit of FIG. 6A, however this time the drill bit is not passed through a transhumeral opening, but instead enters the joint space, passes through a channel in the sizer handle, and through an opening in the glenoid sizer;

FIG. 7A is a perspective side view of the glenoid guide of FIG. 3 coupled to the humeral resection surface of the human glenohumeral shoulder joint of FIG. 6B with a distal end of the glenoid guide disposed in the joint space and the drill bit of FIG. 6B being engaged with the glenoid guide and passed through the transhumeral opening of FIG. 6A;

FIG. 7B is a detailed perspective side view of the distal end of the glenoid guide of FIG. 7A with the drill bit passing through a distal opening of the glenoid guide and into the glenoid surface, with the humerus and the glenoid illustrated translucently;

FIG. 8A is a detailed perspective view of the distal end of the glenoid guide of FIG. 7A with the drill bit passing through a distal opening of the glenoid guide and into the glenoid surface and being used in conjunction with the glenoid sizer of FIG. 6B, the glenoid sizer being disposed above the drill bit;

FIG. 8B is a detailed perspective view of the distal end of the glenoid guide of FIG. 8A with the glenoid sizer being centered on the glenoid and having the grill bit disposed therethrough;

FIG. 9A is a bottom perspective view of another embodiment of a glenoid sizer, illustrating an exterior surface thereof;

FIG. 9B is a top perspective view of the glenoid sizer of FIG. 9A, illustrating an interior surface thereof;

FIG. 9C is a side perspective view of a cannulated drill guide that can be used with the glenoid sizer of FIG. 9A;

FIG. 9D is a side cross-sectional view of the glenoid sizer of FIG. 9B taken along line D-D, the guide being coupled to the cannulated drill guide of FIG. 9C;

FIG. 9E is a side perspective view of the glenoid sizer and cannulated drill guide of FIG. 9D, prior to the drill guide being coupled to the glenoid sizer, and located in the glenohumeral joint space of FIG. 5A with both the cannulated drill guide and a drill bit being passed through the humerus;

FIG. 10A is side perspective view of the reamer attachment of FIG. 5A placed within the joint space of FIG. 7A and the driver of FIG. 5D entering the joint space towards the reamer attachment;

FIG. 10B is a side perspective view of the driver of FIG. 10A coupled to the reamer attachment of FIG. 10A in the joint space;

FIG. 10C is a side perspective view of the reamer attachment of FIG. 10B driven by the driver and entering the glenoid surface;

FIG. 10D is a side perspective view of the reamer attachment of FIG. 10C entering the glenoid surface of the human glenohumeral should joint, still coupled to the driver;

FIG. 10E is a side perspective view of the reamer attachment of FIG. 10D being removed from the driver;

FIG. 11 is a side perspective view of another embodiment of a reamer attachment placed within the joint space of FIG. 5A, the reamer attachment being used in conjunction with a guidewire inserted into the glenoid;

FIG. 12A is a side perspective view of one embodiment of a guidewire adapter;

FIG. 12B is a bottom perspective view of the guidewire adapter of FIG. 12A;

FIG. 13A is a side perspective view of an alternative embodiment of a glenoid guide, though similar to the glenoid guide of FIG. 3, having an embodiment of an implant coupling effector coupled to a distal end thereof, the implant coupling effector being coupled to a glenoid implant;

FIG. 13B is a perspective view of the human glenohumeral shoulder joint of FIG. 10D with the glenoid guide of FIG. 13A positioning the implant coupling effector and the glenoid implant component into the glenoid surface;

FIG. 14A is a side perspective view of the glenoid guide of FIG. 13A having an embodiment of an impactor coupled to the distal end thereof;

FIG. 14B is a perspective view of the human glenohumeral shoulder joint of FIG. 13B with the glenoid guide of FIG. 14A positioning the impactor against the glenoid implant;

FIG. 15 is a side perspective view of one embodiment of a humeral guide coupled to the humeral resection surface of FIG. 2A, the humeral guide including a rigid arm, a hub, support rods, and bone pin clamps, the figure also including a drill cannula associated with the hub, a humeral sizer attachment coupled to the rigid arm, bone pins disposed within the bone pin clamps, and a drill bit disposed within the drill cannula and passing through a transhumeral tunnel and the humeral sizer attachment;

FIG. 16A is a top perspective view of the humeral guide, drill cannula, and humeral sizer attachment of FIG. 15;

FIG. 16B is a top view of the humeral sizer attachment of FIG. 16A aligned and centered against the humeral resection surface of FIG. 15;

FIG. 16C is a perspective view of the humeral sizer attachment and humeral resection surface of FIG. 16B;

FIG. 16D is a bottom perspective view of one embodiment of a sizer plate;

FIG. 16E is a top perspective view of the sizer plate of FIG. 16D as it is removed from the humeral sizer attachment of FIG. 16C and from the joint space;

FIG. 16F is a top perspective view of another sizer plate being coupled to the humeral sizer attachment of FIG. 16C;

FIG. 17 is a side perspective view of the humeral sizer attachment of FIG. 16C as it is removed from the joint space; and

FIG. 18 is a side perspective view of another embodiment of a humeral guide fixed to the humeral resection surface of FIG. 2B and being used in conjunction with the glenoid sizer of FIG. 9A.

### DETAILED DESCRIPTION

Certain embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting embodiments and that the scope of the present disclosure is defined solely by the claims. The features illustrated or described in connection with one embodiment may be combined with the features of other embodiments. Such modifications and variations are intended to be included within the scope of the present disclosure. Accordingly, aspects and features of every embodiment may not be described with respect to each embodiment, but those aspects and features are applicable to the various embodiments unless statements or understandings are to the contrary. Further, to the extent portions of a human anatomy are illustrated, but other portions of the anatomy in that same region are not explicitly illustrated, a person skilled in the art will appreciate the location of the omitted anatomies. In at least some instances the omitted anatomies are not included to improve visualization for the provided explanations and illustrations. A person skilled in the art will still understand how the devices and methods provided for herein can interact with such omitted anatomies without requiring specific illustration of the same.

While in some embodiments movement of one component and/or portion of the body is described with respect to another, a person skilled in the art will recognize that other movements are possible. Additionally, a number of terms may be used throughout the disclosure interchangeably but will be understood by a person skilled in the art. By way of non-limiting example, the terms subscapularis, subscapularis tissue, subscapularis tendon, subscapularis muscle, and other variations of the same, may be used interchangeably with one another, and to the extent some such terms do not appear, they are encompassed by use of the others. By way of further non-limiting example, the terms prosthesis and implant may be used interchangeably with one another.

To the extent the present disclosure describes “coupling,” “mating,” or uses other similar terms as it relates to having an instrument or tool contact part of a patient's anatomy, such as bone, the term includes engagement or contact between the instrument or tool and the part of the patient's anatomy, and does not necessarily require any securing or attaching relationship between the two unless otherwise indicated or understood by a person skilled in the art to inherently create a secured/attached relationship and/or for such securement/attachment to be required for proper performance. Further, to the extent that linear or circular dimensions are used in the description of the disclosed devices, components, systems, and methods, such dimensions are not intended to limit the types of shapes or sizes of such devices, components, and systems, etc. A person skilled in the art will recognize that an equivalent to such linear and circular dimensions can be easily determined for any geometric shape (e.g., references to widths and diameters being easily adaptable for circular and linear dimensions, respectively, by a person skilled in the art). To that end, to the extent the term “circumference” is used, a person skilled in the art will appreciate a “perimeter” or “edge” is an equally acceptable term to use to the extent an equivalent of what is being described is not circular. Likewise, to the extent the present disclosure discusses identifying a center or center point, or other similar location (e.g., substantially center), and/or disposing and/or passing an instrument through and/or at a center or center point, or other similar location (e.g., substantially center), a person skilled in the art will appreciate in at least some instances the “center” or “center point” may be substantially center with respect to the referenced surface and/or another location that is not a center can be used, such location being selected by a surgeon based, at least in part, on an anatomy of a patient, the configuration of the instruments and/or tools being used, and/or surgeon preferences, among other factors.

Sizes and shapes of the components of the guides (e.g., glenoid and/or humeral), reamers, drill guides, implants, and related components, instruments, etc. can depend, at least in part, on the sizes and shapes of the other components with which the guides and related components are being used, the anatomy of the subject being operated on, and the type of procedure being performed. Still further, to the extent features, sides, or steps are described as being “first” or “second,” such numerical ordering is generally arbitrary, and thus such numbering can be interchangeable. Similarly, the order in which actions are presented in claims is by no means limiting. For example, a claim that recites an action of removing a glenoid guide from a surgical site, with that action being listed after an action of reaming a glenoid does not mean that it is required that reaming occurs and then the glenoid guide is removed. In at least some embodiments, the glenoid guide can be removed from the surgical site before the glenoid is reamed, even in instances where they ordered differently in the claim. Only if the claim explicitly requires a particular order is that order applicable.

While terms like “proximal” and “distal” as used herein, they are primarily used as a point of reference for describing two portions or ends of an instrument, tool, component, device, system, location in a body, etc. Accordingly no meaning should be attributed to a specific location with respect to “proximal” or “distal” beyond distinguishing one side from another unless explicitly indicated. For example, what is referred to herein as a proximal portion or end may be considered distal in operation, and thus, likewise, what is referred to herein as a distal portion or end may be considered proximal in operation.

In the present disclosure, like-numbered components of various embodiments generally have similar features when those components are of a similar nature and/or serve a similar purpose, unless otherwise noted or otherwise understood by a person skilled in the art. To the extent terms like “approximately,” “about,” and “substantially” are used herein, a person skilled in the art will appreciate the scope those words convey in the context of their usage. During a surgical procedure, obtaining a certain degree of placement, a certain distance, and/or a certain alignment, among other positioning and the like may be difficult, and thus use of terms like “approximately,” “about,” and “substantially” is intended to address this difficulty. A person skilled in the art will understand what constitutes how close a particular dimension or placement should be to still fall within the spirit of the quantification and description provided for herein.

Even in instances where such terminology is not used, and a dimension or placement just includes the number or placement term (e.g., “parallel” is used instead of “substantially parallel”), a person skilled in the art will appreciate that, unless explicitly indicated otherwise, terms like “approximately,” “about,” and “substantially” are applicable to those dimensions and placements as well. The foregoing notwithstanding, a person skilled in the art will appreciate that terms like “approximately,” “about,” and “substantially” at least encompass dimensions that are +10%, 10°, etc. of the provided amount, or encompass dimensions that are +5%, 5°, etc. of the provided amount, unless indicated otherwise or otherwise known to those skilled in the art. The present disclosure appreciates that a person skilled in the art, in view of the present disclosure, understands suitable placements for various features of the disclosed systems, devices, instrumentation, and/or implants, and related components of any of the same, and thus to the extent a particular placement or location is described, unless it is explicitly indicated that placement or location is required, a person skilled in the art will appreciate other placements or locations that are possible without impacting the overall procedure(s).

### Relevant Anatomy for Shoulder Procedures

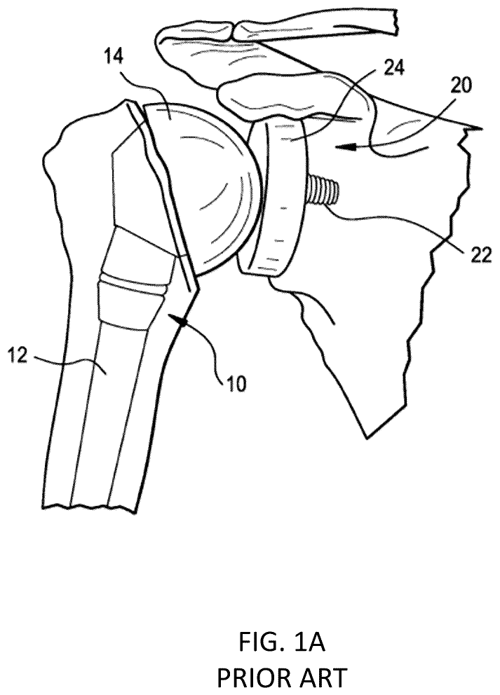

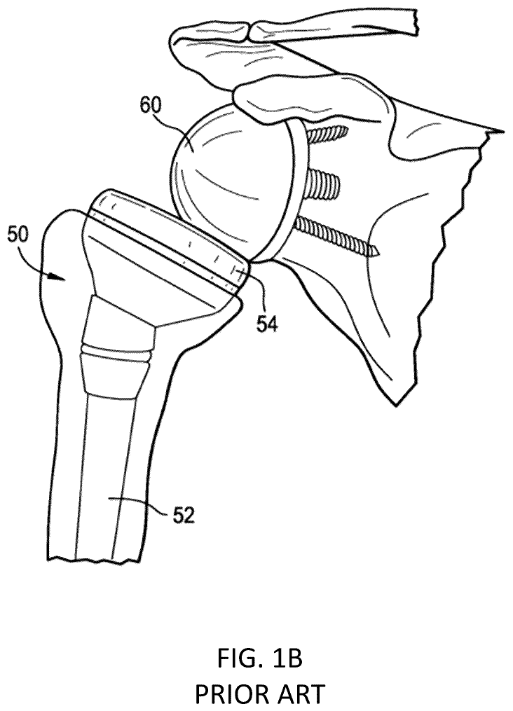

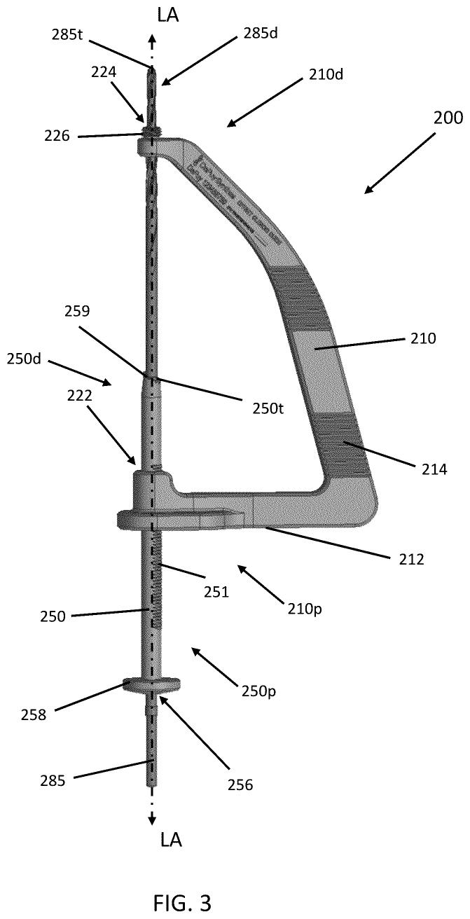

The present disclosure relates to systems, devices, and methods for preparing a surface of a glenoid to receive an implant and/or prosthesis, and more particularly for assisting in reaming a glenoid surface and implanting an implant and/or prosthesis within the glenoid surface. Embodiments of devices and methods disclosed herein can be used in conjunction with a glenoid guide and/or a humeral guide to prepare a surface of a glenoid prior to replacing the surface with a prosthetic, also referred to as a prosthetic implant and/or implant. One non-limiting embodiment of such a guide is described below with respect to at least FIG. 3. The humeral prosthesis or implant can be, for example, a humeral head prosthesis, like the prosthesis 10 of FIG. 1A that includes a convex humeral head 14, which is intended to mimic an anatomically correct humeral head in an anatomic shoulder arthroplasty procedure. The prosthetic convex humeral head 14 is then received by a concave receiver prosthesis, like the prosthesis 24 of FIG. 1A. Alternatively, the humeral implant or prosthesis can be, by way of further example, a concave receiver humeral prosthesis, like the prosthesis 50 of FIG. 1B that includes a concave receiver surface 54, which is intended to mimic a glenoid surface formed on the humerus to receive a prosthetic head component associated with the glenoid, like the convex prosthetic head 60 of FIG. 1B, as used in a reverse shoulder arthroplasty procedure. Although the prosthesis 10 of FIG. 1A includes a stem, as provided for herein, in other instances the equivalent implant or prosthesis to the prosthesis 10 can be stemless.

Whether installing an implant(s) and/or prosthesis(es) for an anatomic or reverse shoulder procedure, the surface of the glenoid must be prepared properly to receive the implant and/or prosthesis securely. Specifically, the surface of the glenoid is typically reamed to a geometry corresponding to the geometry of the chosen implant and/or prosthesis. Guides of the present disclosure enable a surgeon to perform these glenoid surface preparation steps, as well as other steps related to shoulder repair procedures, such as steps like preparing a humeral resection surface and/or implanting one or more implants and/or prostheses. Further, the guides include various features that enable a surgeon to perform these repairs in smaller spaces, such as procedures performed using tissue sparing techniques that do not detach the subscapularis tendon from its natural attachment points. In other words, the subscapularis remains intact during the procedure. An example in which a guide is employed in a tissue sparing procedure can include the guide being fixed to a humerus to guide drilling of a bone tunnel from a lateral cortex, also referred to as a lateral surface, of the humerus exiting substantially central and substantially perpendicular to the humeral resection surface, referred to herein as a transhumeral bone tunnel. Preparation steps such as reaming a glenoid surface can be accomplished through the transhumeral bone tunnel. Locked engagements between a guide pin passed through the bone tunnel and the attachments placed in the joint space can provide the required force for such surface preparation steps. The same guide can be utilized to further guide preparation of the glenoid surface.

The present disclosure provides for tissue sparing procedures to be performed in which the subscapularis tendon remains intact throughout the procedure. Maintaining attachment of the subscapularis tendon means there is more limited space to perform procedures, and the devices, tools, and systems disclosed herein allow for the same types of procedures to be performed (e.g., shoulder arthroplasty) while causing less harm and damage to tissue and the surrounding anatomy. In some embodiments, such as when a tight joint is involved, a portion of the subscapularis tendon may be cut or sacrificed to increase access to the joint. This may entail, for example, cutting a top of the subscapularis tendon a few millimeters, with it being able to be sutured back after having gained sufficient access to perform the procedure(s). By way of further example, about 10% or less of the subscapularis may be sacrificed while about 90% or more of the subscapularis can remain intact. Instances where a portion of the subscapularis tendon, and/or other tendons, are cut or sacrificed can still be considered tissue sparing with respect to the present disclosures, and are still considered to involve the subscapularis being intact. In other words, “intact” does not have to mean fully intact, it can include anything less than fully intact and/or can encompass the subscapularis tendon being “substantially intact,” where “substantially intact” can include any instance when 20% or less of the subscapularis is scarified, or when 15% or less of the subscapularis is scarified, or when 10% or less of the subscapularis is scarified.

As provided for herein, at least two different techniques can be employed to treat the glenoid surface-one that uses a guidewire and another that does not. Variations of guidewire and non-guidewire techniques are also provided for herein. For example, during at least some tissue sparing arthroplasty procedures, the humerus can be rotated upwards and a guide can be mounted to the humerus and used to guide a guide pin or guidewire into a center point of the glenoid surface (which can, in the alternative, be substantially center to the glenoid surface or another location as desired) through a transhumeral bone tunnel. The guide pin or guidewire can then remain fixed in the glenoid and serve as a guide for one or more reamer attachments to carve or otherwise form the desired geometry in the surface of the glenoid. Alternatively, and with the humerus rotated upwards and the guide mounted to the humerus, a driver, such as one having a bullet tip as described herein, can be used to help guide the reaming actions such that no guidewire is disposed at the surgical site and/or gets disposed in a portion of the glenoid.

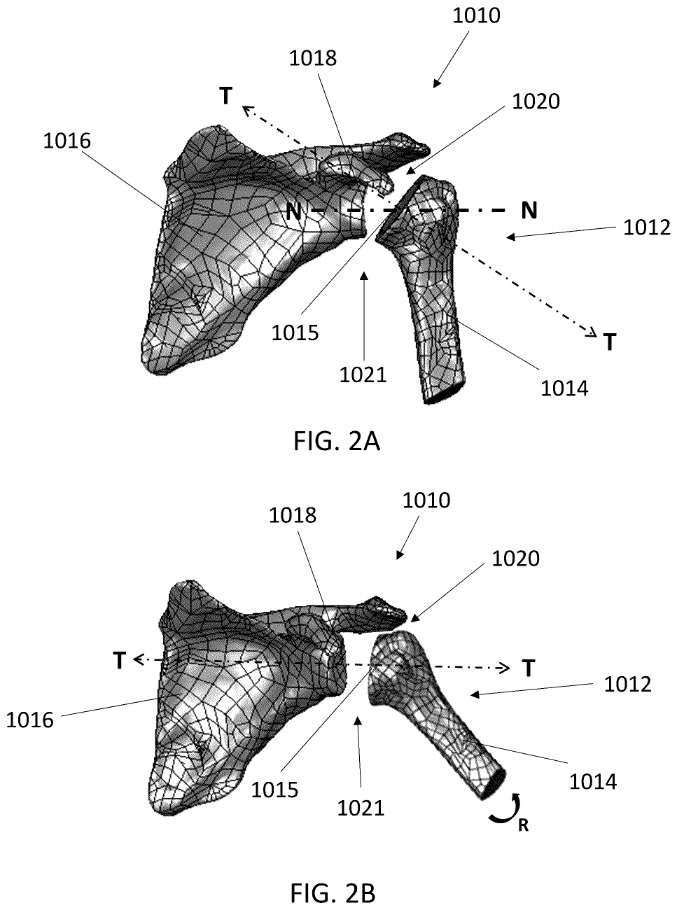

A patient's resected glenohumeral joint 1010, which is part of a shoulder region of a patient, is illustrated in FIGS. 2A and 2B. The glenohumeral joint 1010 is also referred to herein as the joint space 1010 or shoulder joint 1010, among other names. The natural shoulder joint 1010 includes a humerus 1012 and a scapula 1016. The humerus 1012 includes a head portion (shown resected to form a humeral resection surface 1015, but a person skilled in the art knows what a non-resected head portion looks like as well), an elongate shaft 1014, and a lateral cortex 1023, also referred to as a lateral surface in some instances. The humeral resection surface 1015 has a generally circular cross-sectional area, although in other instances it can be considered elliptical or other shapes. The scapula 1016 includes a concave surface or glenoid 1018. During movement of the shoulder joint 1010, the humeral head articulates within the glenoid 1018 of the scapula 1016. If the natural shoulder joint 1010 suffers a traumatic injury or degenerative changes, a surgeon may replace either or both the naturally convex humeral head and the glenoid 1018 with prosthetic components, using either an anatomic or reverse procedure, as detailed above. Although not illustrated, a person skilled in the art will understand that a subscapularis tendon extends from the scapula 1016 to the humerus 1012, and that in a tissue sparing procedure in which the subscapularis tendon is not detached from the humerus (i.e., the subscapularis tendon remains intact), the subscapularis tendon can be manipulated to improve visibility to a surgical site, such as by using displacement wrap that is disposed around at least a portion of the subscapularis tendon and is tensioned in a manner that causes the tendon to be moved inferiorly or superiorly. Non-limiting examples of such a wrap are disclosed in U.S. Patent Application Publication No. 2024/0108433, entitled “Devices and Methods for Minimizing Damage to Soft Tissue during a Surgical Procedure,” the content of which is incorporated by reference herein in its entirety.

Additional tools, such as a double bent Hohmann retractor(s), posterior cuff retractor(s) (e.g., twisted Hohmann retractor(s)), anterior subscapularis retractor(s) (e.g., right angle Hohmann retractor(s)), inferior subscapularis retractor(s) (e.g., subscap Hohmanns retractor(s)), and/or other types of retractor(s) (e.g., double bent Hohmann retractor(s)), including retractors designed for use from a right side and/or a left side of a patient, can be used to further manipulate surrounding soft tissues. Even when a displacement wrap and/or other tools are utilized, however, the amount of space created is typically insufficient for traditional bone preparation instruments and/or tools to be used to perform various bone preparation actions. In such instances, typically a rotator interval 1020, which can be defined between the superior border of the subscapularis and the anterior border of the supraspinatus, provides a first, superior entry point for accessing the humeral resection surface 1015, and an inferior border 1021 of the subscapularis, which can be defined as the lower inferior border of the subscapularis at the lever of the anterior circumflex vessels (i.e., the “Three Sisters”), provides a second, inferior entry point for accessing the humeral resection surface 1015.

In addition to the disclosed procedures being able to be performed while keeping the subscapularis intact and/or without resecting the subscapularis, and the present devices and systems allowing for the same, the present disclosure also allows for the disclosed procedures to be performed without having to externally rotate the humerus to allow access to the joint space. To the contrary, the present procedures, devices, and systems enable the humerus to not be distracted from its joint during the surgical techniques provided for herein.

As shown, the humeral head is typically removed or resected as part of preparing the surgical site to receive an implant and/or prosthesis, leaving a flat planar humeral resection surface 1015. The resection can be performed using a humeral resection guide, also referred to as a humeral cut guide, among other terms, designed for use in tissue sparing procedures. Non-limiting examples of such a cut guide are disclosed in U.S. Provisional Patent Application No. 63/579,942, entitled “Humeral Cut Guides, and Related Methods, for Use in Tissue Sparing Shoulder Arthroplasties,” filed Aug. 31, 2023, as well as U.S. non-provisional patent application entitled “Humeral Resection Guides, and Related Methods, for Use in Tissue Sparing Shoulder Arthroplasties,” filed on an even date herewith, the content of each which is incorporated by reference herein in its entirety.

Traditional tools to prepare the glenoid surface for receiving an implant and/or prosthesis rely on adequate visibility and access to the joint space provided by removing the subscapularis tendon and externally rotating the humerus 1012. As a result, and further aided by the humeral head being resected, a surgeon can have full visibility to the glenoid surface that is being treated to receive an implant and/or prosthesis. With the adequate space, the surgeon can have improved visibility and can impart the necessary forces needed to ream or otherwise create a geometry on the surface of the glenoid 1018 that corresponds to the chosen implant and/or prosthesis. For tissue sparing procedures, as well as other types of procedures performed in more limited space and/or with more limited displacement of tissue and the like, devices like a guide like those provided for herein and those incorporated by reference herein, are helpful.

Further, even when a displacement wrap is utilized, the amount of space created is typically not sufficient for traditional glenoid preparation methods. In such instances, typically a rotator interval 1020 provides a first, superior entry point for accessing the glenoid 1018, and an inferior border 1021 of the subscapularis provides a second, inferior entry point for accessing the glenoid 1018. The aforementioned guide, which again can have a variety of configurations, can provide a third, central entry point to the glenoid 1018 through a transhumeral bone tunnel, as shown in FIG. 2B. More specifically, the transhumeral bone tunnel can be formed in the humerus 1012 along a longitudinal axis T, for example by passing a drill therethrough. The drill can be aligned at the desired orientation and position to form the transhumeral bone tunnel by way of a guide, as described below. As shown comparing FIGS. 2A and 2B, in at least some instances, the humerus can be manipulated to align that tunnel with a location at which drilling is to be performed on and through the surface of the glenoid 1018. More specifically, the trajectory can be adjusted by rotating the arm upward in the R direction until the axis T is aligned substantially perpendicular and center to the surface of the glenoid 1018. A variety of techniques can be used to maneuver the humerus 1012 in this way, including but not limited to utilizing external arm positioners to position the arm and the axis T in the position illustrated in FIG. 2B. In an alternative approach, minimal to no trajectory adjustment may be necessary or appropriate. For example, a transhumeral bone tunnel can be created with the patient arm in a more neutral adducted position, as depicted in FIG. 2A by way of the line N-N.

In addition to the disclosed procedures being able to be performed while keeping the subscapularis intact and/or without resecting the subscapularis, and the present devices and systems allowing for the same, at least some embodiments of the present disclosure allow for the disclosed procedures to be performed without having to externally rotate the humerus to allow access to the joint space. To the contrary, at least some of the present procedures, devices, and systems enable the humerus to not be distracted from its joint during the surgical techniques provided for herein.

Although the present disclosure is often described herein as being applicable to tissue sparing procedures in which the subscapularis tendon remains intact, a person skilled in the art will appreciate that the devices, systems, and techniques described herein can also be used in conjunction with procedures in which the subscapularis tendon is detached from the humerus.

Prior to introducing the guide, the glenoid can be exposed and the labrum can be removed using techniques known to those skilled in the art. This can involve, for example, the use of one or more glenoid retractors placed to allow desired access and/or visualization of the surgical site. Glenoid sizers, at least some examples of which are provided for herein, can be used to size the implant and a glenoid drill can be used to spot face an approximate center of the glenoid, as also provided for herein. The sizing and formation of a central hole, or approximately central hole, in a surface of the glenoid can be used to guide transhumeral glenoid preparation, as also provided for herein.

Generally, the disclosed glenoid guides are fixed to a humerus to guide drilling of a bone tunnel from a lateral surface of the humerus 1012, e.g., the lateral cortex 1023, exiting substantially central and substantially perpendicular to the humeral resection surface 1015, referred to herein as a transhumeral bone tunnel. Preparation steps such as reaming a glenoid surface can be accomplished through the transhumeral bone tunnel. Locked engagements between a guide pin passed through the bone tunnel and the attachments placed in the joint space can provide the required force for such surface preparation steps. The same guide can be utilized to further guide preparation of the glenoid surface similar to the humeral resection surface 1015.

The surgical techniques provided for herein generally have a transhumeral approach, meaning the approach involves coming from a location that is lateral to the humerus. More specifically, in at least some instances, as provided for herein, the transhumeral approach can occur from a location that allows for a surgical access hole that is orthogonal, or substantially orthogonal, to a plane of the humeral resection surface 1015.

### Glenoid Guide(s)

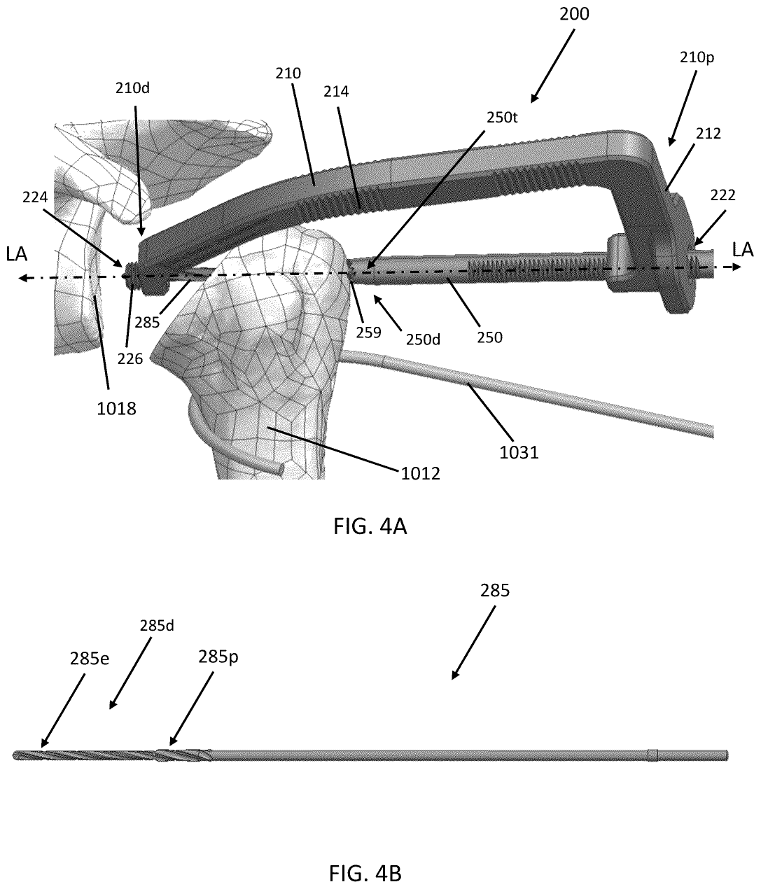

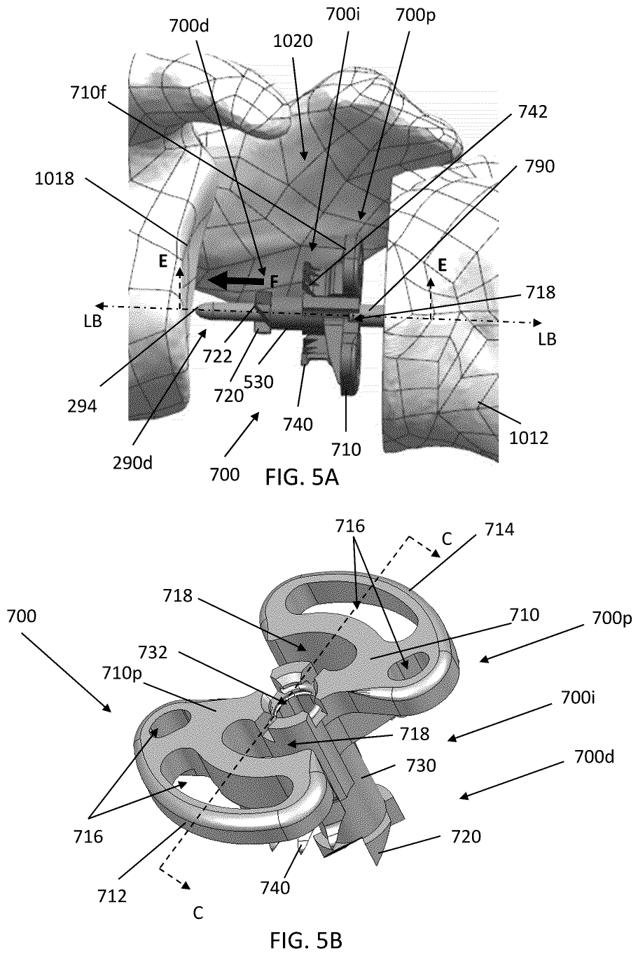

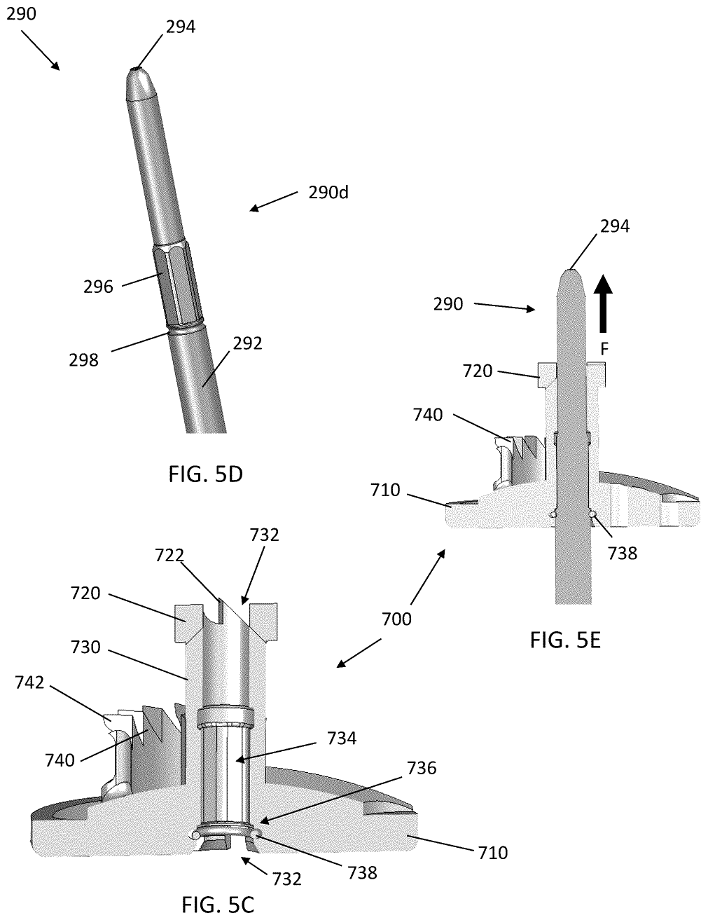

FIGS. 3 and 4A illustrate an embodiment of a glenoid guide 200, also referred to as a glenoid guide frame or an offset glenoid guide in view of an arm thereof having an offset configuration, and FIGS. 5A-5E illustrate components, such as a reamer attachment 700 and driver or drive shaft 290, that can be used in conjunction therewith. Glenoid guides such as the guide 200 can include a rigid, offset arm 210 with a distal portion 210d and a proximal portion 210p. The rigid arm 110 can be sized and shaped to allow for proper centering and alignment between, for example, a cannulated bullet or drill cannula 250 coupled to or otherwise associated with proximal portion 210p of the arm and a center of the glenoid surface 1018, as shown in FIG. 4A. The size and/or shape of the arm 210 can depend, at least in part, on the size and anatomy of the patient (e.g., child vs. adult, male vs. female, etc.) and/or the preferences of the surgeon. In the illustrated embodiment, the length of the rigid arm 210 includes curved or arcuate shape as it extends from the proximal portion 210p of the arm 210 to the distal portion 210d of the arm 210, terminal ends thereof defining the length of the arm 210. In a variety of embodiments, including in the one shown, the rigid arm 210 can be sized and shaped to allow for the glenoid guide to be able to pass into the shoulder joint 1010 in a minimally invasive manner. Further, in a variety of embodiments, including in the one shown, the ridged arm 210 can be sized and shaped to allow for grasping with one hand during a surgical procedure, which in turn can provide for universal adaptation such that it can be grasped in an equally convenient and easy-to-use manner by a user's right hand or left hand without having to change grips and/or positions during the surgical procedure. Accordingly, the glenoid guide 200 can be considered a universal glenoid guide.

In the illustrated embodiment, a first, proximal opening 222 and a second, distal opening 224 are formed in respective proximal and distal ends of the proximal and distal portions 210p, 210d of the arm 210. The proximal opening 222 can be considered a cannula-receiving opening as it can first receive a distal end of a drill cannula (e.g., the drill cannula 250) as the drill cannula is inserted into and through the guide 200, as described in greater detail below, while the distal opening 224 can be considered an alignment opening as it can be proximate to a location at which the distal end of the distal portion 210d of the arm 210 is placed so the guide 200 is properly positioned with respect to the glenoid surface 1018. The glenoid guide 200 can be sized and shaped such that the distal opening 224 and cannula-receiving opening 222 align along a same longitudinal axis LA. As such, the distal and proximal openings 224 and 222 can be considered to be colinear. The proximal end of the arm 210 can include a flat surface 212 that is substantially perpendicular to the longitudinal axis LA. The flat surface 212 can be configured to engage with a user's hand or a tool such as a hammer or mallet, for example to provide a force in a direction towards the surgical site (i.e., towards the glenoid surface 1018). The applied force can be passed through the arm 210 and to the distal end of the guide 210, and thus to any other component associated therewith, such as an end effector or implant coupled to the distal end.

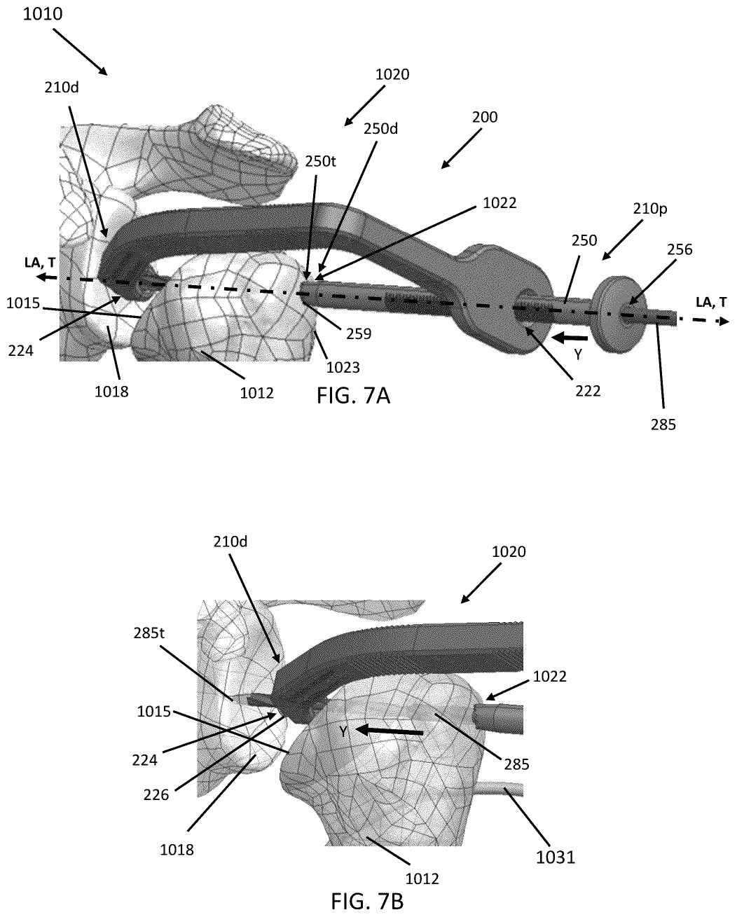

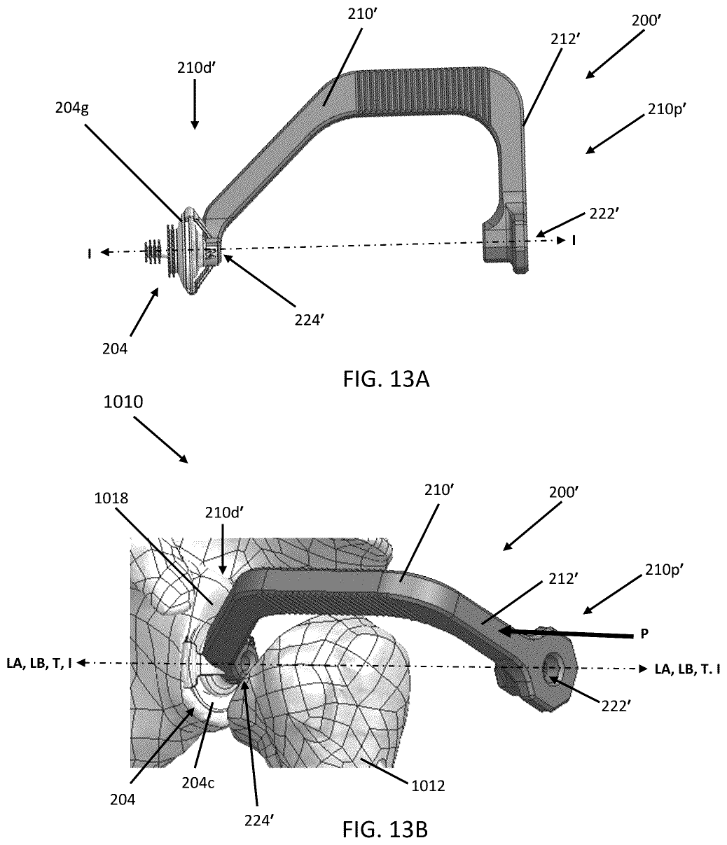

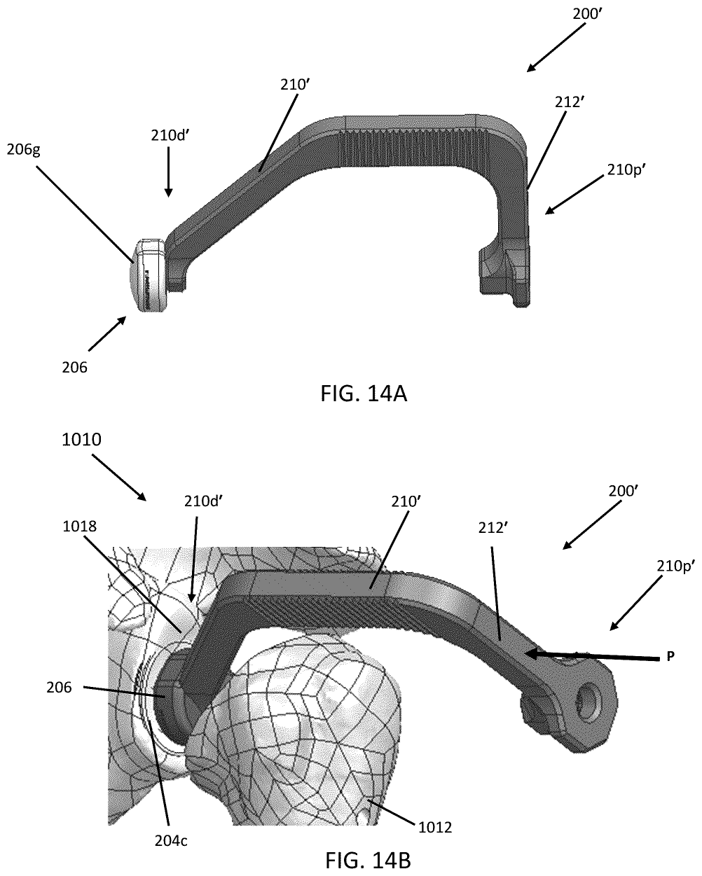

The distal end of the distal portion 210d can include a post 226 that extends from the distal end along the longitudinal axis LA. The distal opening 224 can extend through the post 226 such that the distal end of the arm 210 includes the post 226 with the post 226 being integrally formed with a body of the arm 210, although in other embodiments the post 226 can be a component that is removable and replaceable with respect to body of the arm 210. Further, the opening 224 and post 226 can be configured to receive and mate with an end effector, such as an impactor (see, e.g., FIG. 14A), or implant (see, e.g., FIG. 13A), and/or can help stabilize the glenoid guide 200 with respect to the glenoid surface 1018 (see, e.g., FIG. 7A). In the illustrated embodiment, the post 226 is threaded, although other mating features for purposes of mating with an end effector or implant (e.g., snap-fit, male-female engagement mechanisms, etc.) can be used. As shown, an outer surface of the arm 210 can include gripping features 214 formed thereon. In the illustrated embodiment, the gripping features 214 are ridges, though other gripping features can be used in lieu of, or in addition to, the ridges. The post 226 can be used for implant placement and impaction as desired.

An intermediate portion of the arm 210 that extends from the proximal portion 210p to the distal portion 210d can be offset with respect to the longitudinal axis LA. In particular, as illustrated in use, the longitudinal axis LA can extend through a humerus when coupled to it while the offset arm can extend around the humerus.

The rigid arm 210 can operate in conjunction with the drill cannula 250 to define the location and trajectory at which a bone tunnel is to be drilled into the humerus 1012, and at which the glenoid 1018 will be contacted in conjunction with the various procedures described herein. In the present embodiment, the drill cannula 250 is an elongate, substantially cylindrical or tubular shaft having a base or handle 258 located at a proximal end of a proximal portion 250p of the drill cannula 250, a distal end or tip 250t of a distal portion 250d of the drill cannula 250 configured to engage with bone, and an intermediate portion or length extending therebetween. As shown, ratcheting teeth 251 can be disposed on an outer surface of the intermediate portion of the drill cannula 250. The drill cannula 250 further includes an opening 256 extending through an entirety of a length of the drill cannula 250. The opening 256 can be sized and shaped to allow a drilling component, such as a drive shaft or drill bit 285, also referred to as a driver, to pass through the drill cannula 250 and into the bone in which a bone tunnel is to be formed. The terms drill bit and drill may be used interchangeably herein. As shown, the distal tip 250t can be tapered, for instance by varying a thickness of a wall of the drill cannula 250, to make it easier to push the drill cannula 250 through soft tissue and against the surface of the humerus. In some embodiments, bone engaging features, such as teeth 259, can be formed at the distal tip 250t. The teeth 259 can help stabilize the location of the distal tip 250t of the drill cannula 250 with respect to the bone. The stabilization can provide more accurate trajectory for the bone tunnel. The cannula-receiving openings 222, 224, along with the drill cannula 250, sets the location and trajectory of a bone tunnel or bore to be drilled through the humerus 1012 from the lateral cortex 1023 to the humeral resection surface 1015 and further to the glenoid surface 1018, the tunnel center being substantially perpendicular to the glenoid surface 1018. As shown, the drill cannula 250 passes through and is selectively held within the proximal opening 222 by the guide 200.

Once the glenoid guide 200 is placed proximal the humerus with the distal opening 224 of the arm 210 marking the center of the glenoid surface 1018, the distal portion 250d of the drill cannula 250 can be passed through the cannula-receiving openings 222, 224 and pressed against the lateral cortex 1023 of the humerus 1012 to mark proximal end of the intended bone tunnel and define the entry location for the drilling component. The wall of the arm 210 forming the cannula-receiving opening 222 can be configured to allow for a sliding friction bit between the drill cannula 250 and the guide 200. The ratcheting teeth 251 formed on the outer surface of the intermediate portion of the drill cannula 250 correspond to a one-way ratchet mechanism on the inner surface of the cannula-receiving opening 222 to maintain the position of the drill cannula 250. The ratcheting teeth 251 allow the drill cannula 250 to pass distally through the cannula-receiving opening 222 but prevents the drill cannula 250 from moving proximally thereby maintaining force on the surface of the bone at the distal tip 250t of the drill cannula 250. The drill cannula 250 passes through the cannula-receiving opening 222 until the distal tip 250t meets the lateral surface of the humerus 1012, as shown the lateral cortex 1023.

A drill bit, shown as drill bit or drill 285, can be passed through the drill cannula 250 to create the transhumeral bone tunnel following the trajectory set by the glenoid guide 200. A proximal end can include a connection, which can include a modified trinkle connection, that can be connected to a power source and used to rotate and/or advance a distal end 285d of the drill bit 285, including at a distal tip 285t. Once the bone tunnel has been created, guide pins or drivers with various attachment features at their distal tip can be passed through the drill cannula 250 and attached to various modular attachments inserted into the rotator interval. Embodiments of such guide pins or drivers and their attachment features are described below and/or are incorporated by reference herein and/or are otherwise known to those skilled in the art.

As shown in FIG. 4A, the glenoid guide 200 is employed during tissue sparing arthroplasty surgery after the humeral head has been resected. The distal portion 210d of the arm 210 can be inserted superior to the subscapularis and aligned facing the center of the glenoid surface 1018. The proximal end 210p of the arm can be placed proximal to the lateral cortex 1023 of the humerus 1012 to guide the drill cannula 250 until the distal end of the drill cannula 250 meets the lateral cortex 1023 of the humerus 1012, setting the location and trajectory for a transhumeral bone tunnel. More particularly, the glenoid guide 200 ensures that a trajectory for a guide pin or drill passed through the drill cannula 250 substantially central and substantially perpendicular to a surface of the glenoid 1018. A bone hook 1031 is illustrated in FIG. 4A, and as described below with respect to glenoid preparation methods, can be used for lateral humeral manipulation to increase visualization within the joint.

The drill bit 285 can contact and enter the glenoid surface 1018 to form a pilot hole in the glenoid. In the illustrated embodiment, as shown in FIG. 4B, the drill bit 285 can have a plurality of diameters and be referred to as a stepped drill bit or a stepped glenoid drill bit. The configuration can be such that a distal-most end 285e of the distal portion 285d can have a smaller diameter, such as a diameter of about 3.2 millimeters, and a more proximal portion 285p of the distal portion 285d can have a larger diameter, such as a diameter of about 4.0 millimeters. The smaller diameter can be used to form a pilot hole in a glenoid surface, while the larger diameter can be used to form a transhumeral bone tunnel formed in the humerus 1012, or expand a size of such a tunnel initially formed by the smaller diameter. A length of the smaller diameter typically, although not exclusively, can be such that the larger diameter is not involved in forming the pilot hole. In other embodiments, a drill having a single diameter, or even a drill having three or more diameters, can be used to form a pilot hole in the glenoid and/or a transhumeral tunnel formed in the humerus 1012. Still further, in some embodiments, multiple drill bits, the multiple drill bits being of different diameter(s), can also be used in lieu of a single stepped drill bit.

The trajectory of the drilled transhumeral bone tunnel can be extended into the center point of the surface of the glenoid 1018 to accurately and evenly prepare the surface, and the glenoid 1018 more generally, to receive a prosthesis. The center point of the surface of the glenoid 1018 can be visually spotted by the surgeon or a glenoid guide can be used to guide a drill substantially perpendicular and substantially center into the bicortical layer of the glenoid surface creating a pilot hole. In some instances, a guide wire can remain along the axis L in the joint space with the distal end fixed within the bicortical layer for the remaining preparation steps. In other instances, no guide wire is used.

### Glenoid Reamer(s)

After the center point of the surface of the glenoid 1018 is defined, one or more instruments can be introduced to the joint space to treat or otherwise prepare the glenoid 1018 to receive a prosthesis. One such instrument is a glenoid reamer, which can be used to carve out a geometry in the surface of the glenoid 1018 that corresponds with the geometry of the chosen prosthesis. Reaming during a tissue sparing shoulder arthroplasty can be accomplished by providing force to a reamer tool onto the glenoid surface through the transhumeral bone tunnel.

FIGS. 5A-5E illustrates one embodiment of a reamer attachment or reamer 700, and/or components with which the reamer attachment 700 can be used, configured to carve a geometry in a surface of the glenoid 1018. Reaming a bone requires a sufficient amount of pressure applied to the bone surface by the reamer to carve the proper geometry. In traditional methods that were performed using known tools, the narrow rotator interval maintained by the surrounding soft tissue did not provide adequate space to ream the bone surface using a force applied down onto the glenoid surface 1018. Accordingly, the present disclosure provides the required force to operate the reamer by way of a force provided to the reamer attachment 700 by way of a driver 290, also referred to as a bullet tip glenoid drive shaft or a bullet tip driver, passed through the drill cannula 250 and transhumeral bone tunnel. FIG. 5A illustrates the reamer attachment 700 disposed on the driver 290 and disposed in the joint space along the axis LA.

As shown in FIG. 5B, the present embodiment of the reamer attachment 700 includes a base 710 formed at a proximal end 700p of a body of the reamer attachment 700, pilot blades or cutting surfaces 720 formed at a distal end 700d of the body of the reamer attachment 700, and a central tunnel 730 having reaming blades or cutting surfaces 740 disposed therearound along an intermediate portion 700i of the body of the reamer attachment 700. The base 710 can extend between a proximal-most surface 710p (FIGS. 5A and 5B) and a reamer face 710f (FIG. 5A), which is opposed to the proximal-most surface 710p, the proximal-most surface 710p also being the proximal-most surface of the reamer attachment 700. In the illustrated embodiment, the base 710 has a partial circle shape, more particularly having opposed wings 712, 714 disposed symmetrically around a central opening 732 that extends through the body, including through the central tunnel 730, of the reamer attachment 700. Other shapes of the base are possible, including embodiments that have additional wings and embodiments that do not include wings. A plurality of relief holes or openings 716 can be formed in the base 710, extending from the proximal-most surface 710p to the reamer face 710f, the relief holes 716 providing paths for cut tissue, fluids, debris, and other materials to pass through during operation of the reamer attachment 700. As shown, there are four relief openings 716, at least some of which have different sizes and shapes, although other configurations, shapes, and number of relief openings can be provided. Still further, opposed channels 718 can be formed in the base 710 and can extend into the intermediate portion 700i of the reamer attachment 700 as part of the central tunnel 730. The channels 718 can provide, for example, relief for bone and/or cartilage materials as the reamer attachment 700 plunges deeper into bone in use. The flat profile of the base 710 allows the reamer attachment 700 to enter the joint space through the narrow rotator interval 1020 above the subscapularis.

The central tunnel 730 can extend distally from the base 710 and towards the distal portion 700d. The central opening 732 can extend through the tunnel 730, allowing instruments like the driver 290 to pass therethrough. Geometries complementary to the instruments to be disposed through the tunnel 730, and thus through the central opening 732, can be formed therein. For example, a portion of an inner surface of the central opening 732 can have a hex shape, complementary to a hex shape formed in an instrument that passes therethrough.

The reaming blades 740 can be disposed around the central tunnel 730, radially outward from the central opening 732, extending from each of the wings 712, 714. The blades 740 can be sized and shaped to form any number of configurations in the surface of the glenoid 1018, including but not limited to form a concave, smooth surface when rotational force is applied to the reamer 700 and the blades 740 contact the bone. As shown, teeth 742 provided at distal ends of the blades 740 can extend radially outwards at a distal tip thereof, although other teeth configurations are possible.

The pilot blades 720 formed at the distal end 700d, at a terminal end of the central tunnel 730, can also be disposed around the central tunnel 730, radially outward from the central opening 732. The blades 720 can be sized and shaped to form an initial, smaller diameter hole or other shape in the surface of the glenoid 1018, to help prepare the surface to receive the reaming blades 740. As shown, teeth 722 formed on the blades 720 are substantially flat, not extending radially outwards at a distal tip thereof like the teeth 742, although other teeth configurations are possible. After the blades 720 form a pilot hole, the blades 740 engage the surface of the glenoid 1018 to enlarge the previously created pilot hole when rotational force is provided to the reamer attachment 700. Particularly, in at least some embodiments, when a rotational force is provided to the reamer attachment 700, the blades 720 on a distal end of the central tunnel 730 and the blades 740 create ring and post recesses within the surface of the glenoid 1018 to receive an INHANCE™ implant (from Johnson & Johnson of New Brunswick, NJ), although one skilled in the art will recognize that various blade configurations can be placed on the reamer attachment to correspond to varying implant geometries.

The central tunnel 730 and its associated central opening 732 can be configured to accept and secure the driver 290 within as shown in FIGS. 5A and 5E. More specifically, as illustrated in FIG. 5C, the central tunnel 730 extends from the base 710 to the pilot blades 720 with the central opening 732 extending therethrough. A portion 734 of an inner wall of the central tunnel 730 that defines the central opening 732 can be hex-shaped. Further, a snap ring groove 736 can be formed within the opening 732, the groove 736 being configured to receive a snap ring 738 (e.g., a metal snap ring) that extends around the circumference of the central opening 732. In at least some embodiments, the ring 738 can be manufactured into the groove 736 such that it cannot be, or at least not easily be, removed by a user. The ring 738 can provide feedback to a user and/or can resist disassembly, as described in greater detail below.

FIG. 5D illustrates one embodiment of a portion of an instrument with which the reamer attachment 700 can be used. The instrument is a driver 290, and the illustrated portion is a distal end 290d of the driver 290. A person skilled in the art will appreciate the various configurations a proximal end and an intermediate portion of an instrument like the driver 290 can be, and it is thus not necessary to include illustrations or descriptions of the same. As shown, a portion of an elongate shaft 292 of the driver 290 can terminate at a distal end 290d at a rounded distal tip 294, also referred to a bullet-shaped tip or a bullet tip. Further, proximal of the round distal tip 294 can include a hex-shaped portion 296 of the shaft 292. The hex-shaped portion 296 can be complementary to the hex-shaped portion 734 of the central opening 732 formed in the central tunnel 730 of the reamer attachment 700 such that when the hex-shaped portion 296 sit substantially fully with the hex-shaped portion 734, a location of the driver 290 is substantially fixed and held in place with respect to the reamer attachment 700. Although hex-shaped portions 734, 296 are illustrated, a person skilled in the art will appreciate other shapes and configurations that can be used in lieu of hex shapes, and thus these portions can more generally be referred to as keyed portions. When the complementary keyed portions 296, 734 are engaged, the driver 290 can rotate and translate (advance and/or retract) the reamer attachment 700. Additionally, or alternatively, complementary slots and keyways can be formed in the driver 290 and reamer attachment 700 to assist in mating the two components together. An indentation or circular groove 298 can be formed directly proximal of the hex-shaped portion 296. The ring 738 can interact with the groove 298 to provide audible and/or tactile feedback that notifies a user that inserting and seating of the driver 290 into the central opening 732 is complete. The ring 738 and groove 298 can also work together to limit and/or stop further distal movement of the driver 290 in the direction F. Further, the snap ring 738 can provide resistance to disassembly and can keep components assembled to perform reaming. When done with the driver 290, the user can pull back, and the snap ring 738 can expand due, at least in part, to features on an elongate shaft 292 of the driver 290. The shaft 292 can be released and the snap ring 738 can remain in the groove 736.

The reamer attachment 700 can come in different sizes, with a reamer attachment being selected based, at least in part, on the anatomy, age, and other demographics of the patient, along with surgeon preference, among other factors. For example, there may be an extra small, small, medium, large, and extra large sizes, with the larger sizes having larger dimensions for features like the base 710, the pilot blades 720, and the reaming blades 740, among other features.

Returning to FIG. 5A, the reamer attachment 700 can be placed into joint space through the rotator interval 1020. The base 710 of the attachment can be aligned substantially planar to the humeral resection surface 1015. The driver 790 can then be pushed in the direction F such that the distal end 790d of the driver enters the central opening 732 and passes through the central tunnel 430, engaging coupling features between the driver 790 and reamer attachment 700, such as the aforementioned hex-shaped portion 734 and/or the groove 736. The driver 290 can then be further advanced in the direction F with the attachment 700 coupled until the distal tip 294 of the driver 290 enters the previously drilled pilot hole in the center of the surface of the glenoid 1018. The secure connection between the driver 290 and the reamer attachment 700 forces the blades 720, 740 of the reamer attachment 700 into the surface of the glenoid 1018 in response to a rotational force being applied to a proximal end of the driver 290 and therefore the attachment 700. The guidance provided by the driver 290 passed through the transhumeral bone tunnel ensures the plane of the reamer 700 aligns substantially parallel to the plane of the surface of the glenoid 1018 and central to the glenoid 1018 for accurate reaming.

Although the reamer attachment 700 is illustrated, a person skilled in the art will appreciate other actions that can be performed in a similar manner using a different attachment than the reamer attachment. This can include other types of cutting or resecting actions, as well as other ways by which a glenoid surface can be treated for purposes of receiving an implant.

Different sized glenoid guides and reamers, and components thereof, for use with different patient and patient anatomies, and other components of a glenoid guide(s)/reamer(s), and/or used in conjunction with the guide(s)/reamer(s) of the present disclosure, can be provided together as a kit. This glenoid guide kit can include, for example, any combination of arms 210, posts 226, drill cannulas 250, drill bits 285, drivers 290, and/or reamers 700 of various sizes, among other components and features provided for herein. Any other components disclosed or otherwise provided for herein can also be part of such a kit(s). A person skilled in the art will appreciate that such kits are not limited to only the embodiments disclosed and explicitly illustrated herein, but rather, includes various configuration and iterations accounted for in the text and/or otherwise understood to achieve similar purposes as provided for herein. The various components can be sized and/or shaped for different patient anatomies (e.g., adult, child, patient having certain bone formations due to various ailments or diseases, etc.). Further, a glenoid guide kit, and/or components thereof, can be more generally be part of a shoulder arthroplasty surgery kit, or surgical kit more generally.

### Preparing a Glenoid Surface

The glenoid guide, and associated components, can be used, for example, after a humeral head has been resected to form the humeral resection surface 1015. While in some instances it may be appropriate to use a more traditional approach to preparing the glenoid to receive an implant(s), in other instances, a patient anatomy may not allow for guide pin placement and glenoid reaming along a desired glenoid central axis and/or a transhumeral approach may be more preferred for reasons described herein. In at least some such instances, the glenoid guide 200 can be used to achieve a transhumeral approach.

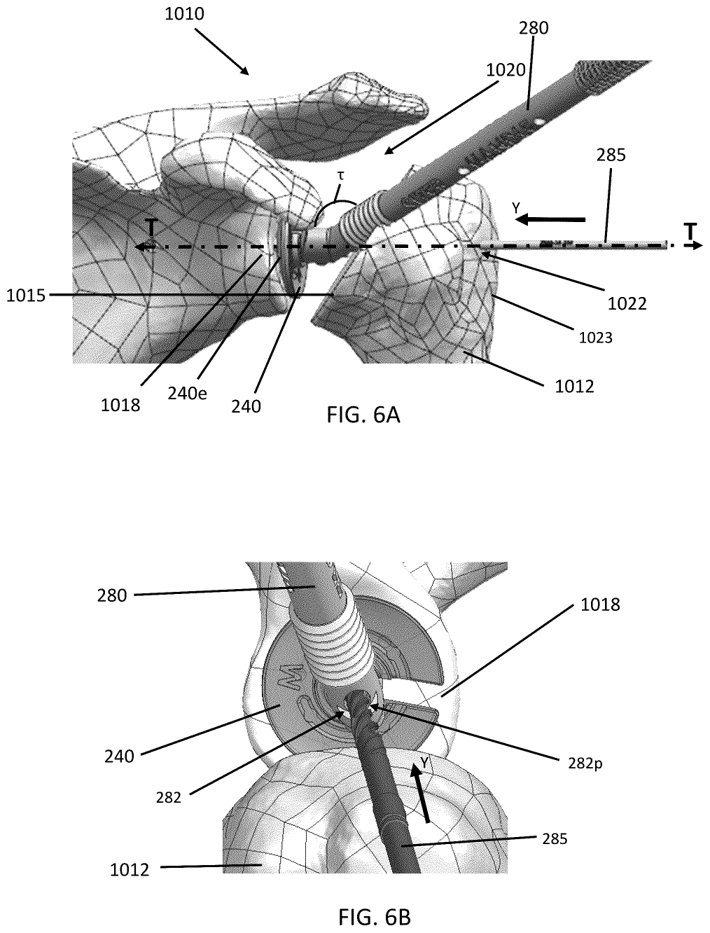

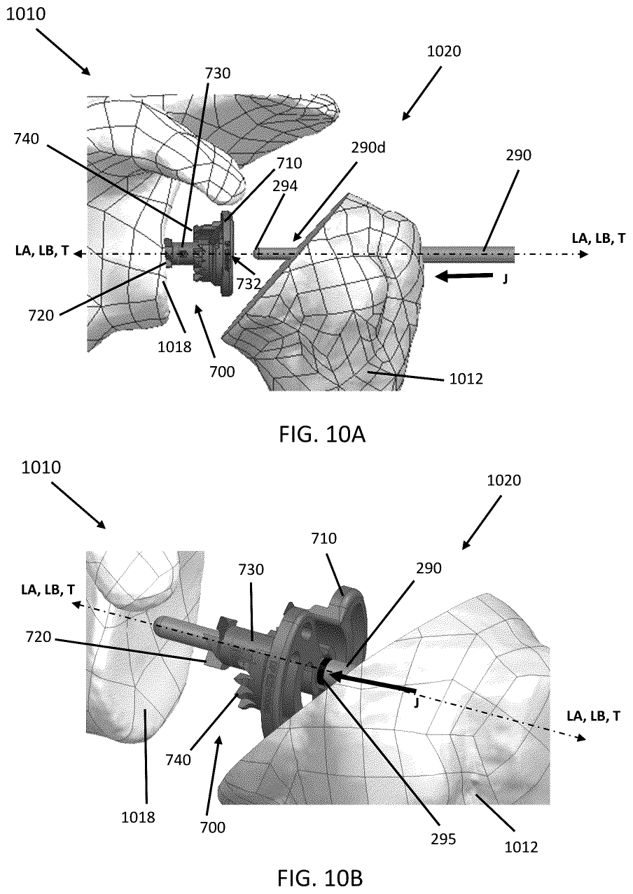

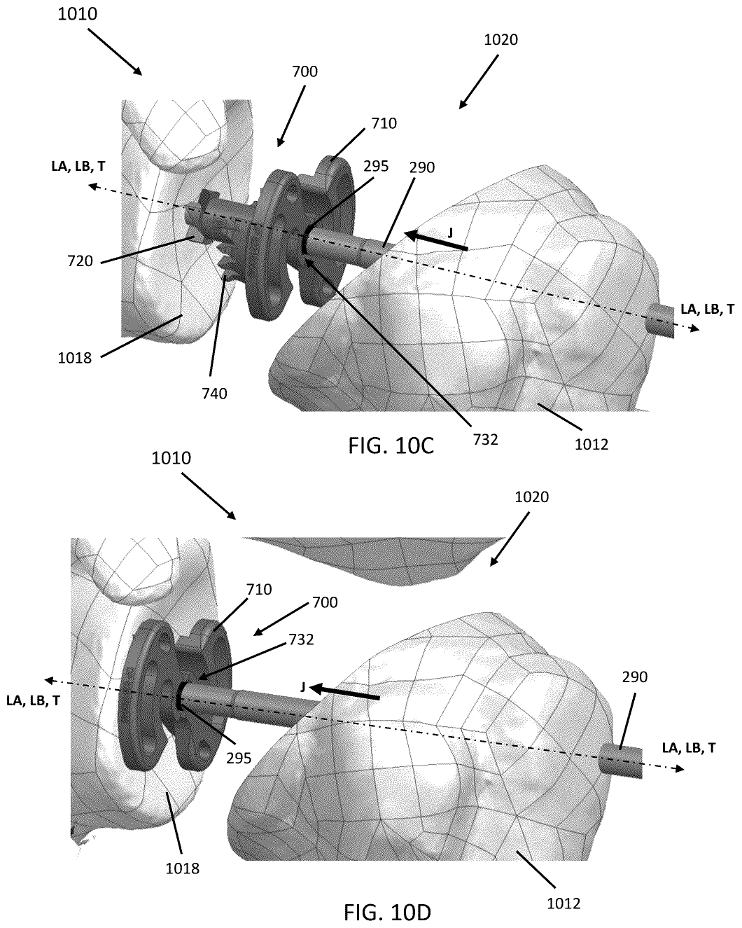

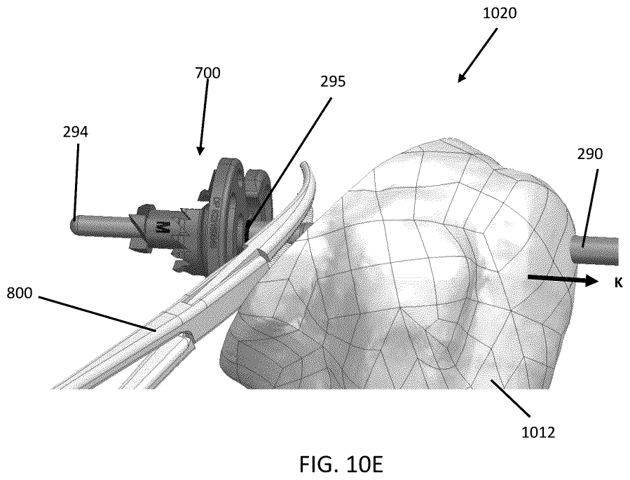

A non-limiting example of a surgical procedure for preparing a glenoid surface to receive an implant and/or a prosthesis using a glenoid guide 200 of the present disclosure is illustrated in FIGS. 6A-8B, with additional aspects of such a procedure described with respect to FIGS. 10-10E and 13A-14B. Prior to introducing a glenoid guide or other glenoid preparation tools to the surgical site, the glenoid can be exposed using techniques known to those skilled in the art and the labrum can be removed or otherwise moved out of the way for access to the surgical site. For example, one or more glenoid retractors can be used for these purposes, as is known to those skilled in the art and described. This may or may not involve using the retractor(s) that were used in conjunction with resecting the humeral head, and to the extent the retractor(s) are used for both, they may or may not be manipulated or otherwise moved to provide desired exposure to the glenoid area of the joint space. In some instances, retractor(s) and related components from the DePuy Global Enable Retractor set (from Johnson & Johnson of New Brunswick, NJ) can be used. These can include an anterior glenoid neck retractor(s) and/or a forked posterior retractor(s). A standard blunt or sharp-tipped Hohmann retractor(s) can be used superiorly. Additionally, use of a bone hook 1031 (see FIGS. 4A and 7B) can be used for lateral humeral manipulation, for instance to increase visualization within the joint.

With the glenoid retractor(s) in place, various instruments can be used to determine the correct glenoid implant size and/or location, and tools such as the glenoid guide 200 can be positioned for use at a surgical site. In the illustrated embodiment, the surgical site is the human glenohumeral shoulder joint 1010 at which the glenoid 1018 and the humeral resection surface 1015 of the humerus 1012 are located, as also illustrated in FIGS. 2A-2B.

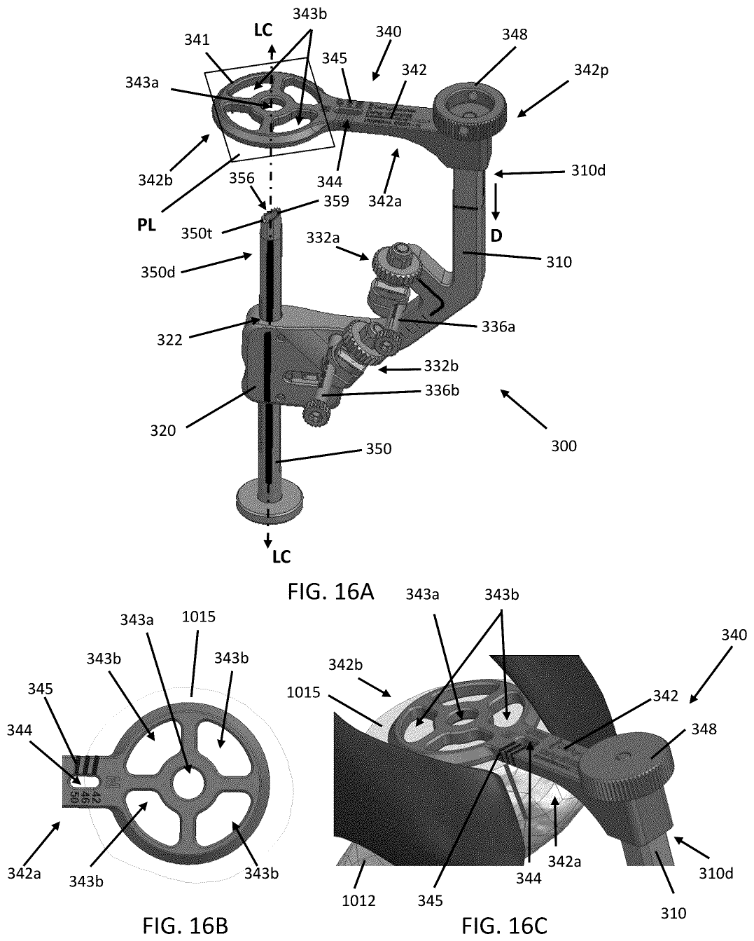

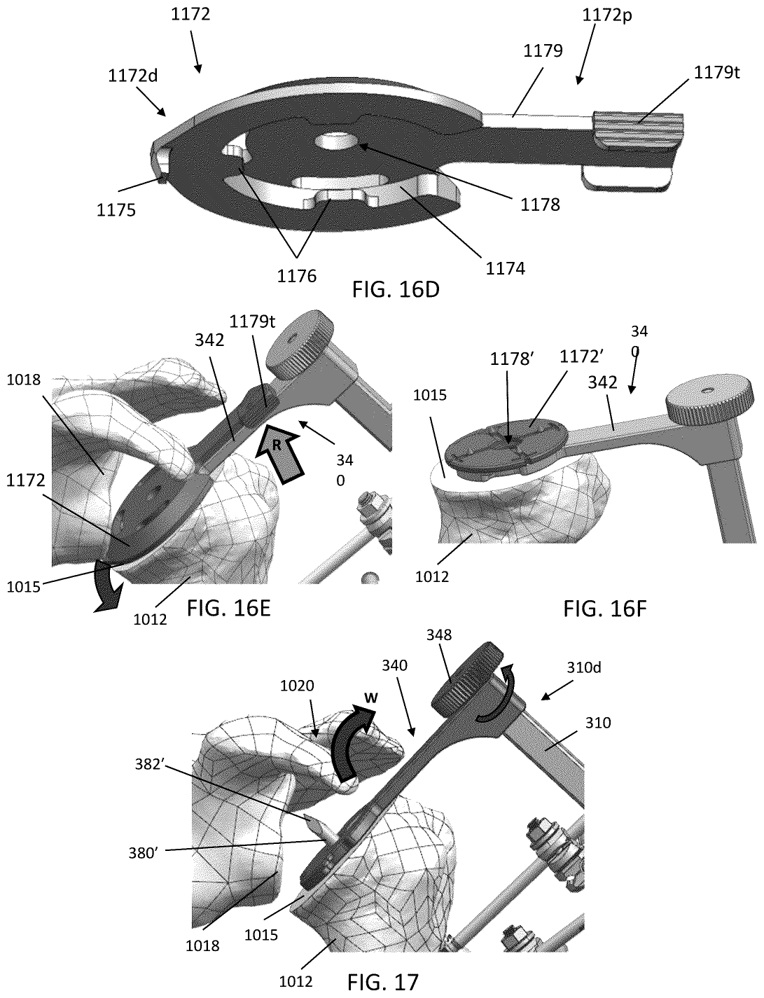

Starting first with determining an appropriate size and location of a glenoid implant, a sizer plate or disc, such as a sizer plate, disc, or sizer 240, which can be one of a plurality of differently sized and otherwise configured sizer plates, can be used for these purposes. The sizer 240 can be introduced into the glenohumeral joint, for example through the rotator interval 1020, to determine the appropriate size of instrumentation and/or prostheses to use at the surgical site and/or to locate the center point or center, or an approximate center point or center, of the glenoid surface 1018. FIGS. 6A and 6B illustrate two manners in which such a sizer 240 can be introduced to the glenoid surface 1018—one in which a transhumeral bone tunnel 1022 is formed in the humerus 1012 (FIG. 6A), and one in which such a tunnel is not (FIG. 6B). The selected sizer 240 can be based on information about the patient, experience of those performing the procedure, determinations made with a humeral sizer attachment (see, e.g., FIGS. 16A-16C), and/or sizers 240 having different sizes that can be introduced into the joint space until the proper size is determined. Non-limiting examples of sizers 240 include INHANCE™ glenoid sizers (from Johnson & Johnson of New Brunswick, NJ). The sizer plate 240 can be attached to a tool, such as a sizer handle 280 as shown in each of FIGS. 6A and 6B, to introduce the plate 240 into the limited joint space through the rotator interval 1020 while keeping surrounding soft tissue intact. As shown, the convex external surface 240e can be aligned against the surface of the glenoid 1018.

As shown in FIG. 6A, in some embodiments, access to a surface of the glenoid 1018 during tissue sparing arthroplasty can be obtained, in part, by drilling a transhumeral bone tunnel 1022 from the lateral cortex 1023 of the humerus 1012, through the humerus 1012, and to the humeral resection surface 1015 such that an axis T of the transhumeral bone tunnel 1022 extends substantially perpendicular and substantially center to the face or surface of the glenoid 1018. As described, for example, with respect to at least FIGS. 10A-10E, any force required to prepare the surface of the glenoid 1018 to receive an implant and/or prosthesis can be provided pushing a driver (e.g., the driver 290) engaged with an attachment (e.g., the reamer attachment 700) placed against the surface of the glenoid 1018 through the transhumeral bone tunnel, thereby forcing the attachment against the glenoid surface 1018. Tools such as the glenoid guide 200 or a sizer handle 280 can be used to set the trajectory and guide the drilling of the transhumeral bone tunnel.

The sizer handle 280 includes a distal end 280d configured to couple to a sizer plate such as the plate 240 and a proximal or handle portion (not shown, but known and understood by a person skilled in the art). The distal end 280d can be joined to the proximal handle portion at a fixed angle t approximately in the range of about 0° to about 60° to allow the sizer plate 240 to sit flush, or substantially flush, with the glenoid surface 1018 while the handle portion 280p extends out of the rotator interval 1020 and further out of the joint space 1010. In the illustrated embodiment, the angle t is about 45°. A person skilled in the art, in view of the present disclosures, will appreciate that a surgeon can flip the handle 280 over and use the long axis of the handle, as it too can be cannulated, which can provide “face-on” access that would be approximately 0°. The illustrated sizer handle 280 further includes a channel 282 extending through the distal portion 280d, as best seen in FIG. 6B. A proximal opening 282p of the channel 282 provides access to the surgical site, i.e., the glenoid surface 1018 as shown, and a distal opening (not visible) of the channel 282 is configured to align with a central opening of the sizer plate 240 when the sizer plate 240 is coupled to the sizer handle 280. This alignment allows for a glenoid drill, such as the drill bit or drill 285, passed therethrough to access the glenoid surface 1018 to form at least a pilot hole therein.

The transhumeral hole or tunnel 1022 can be formed using any techniques known to those skilled in the art for forming a hole or tunnel in bone. For example, the sizer handle 280 and sizer plate 240 can be introduced through the rotator interval 1020, for instance with the subscapularis tendon still intact, such introduction being performed using techniques disclosed herein or otherwise known to those skilled in the art. With the sizer plate 240 centered, or approximately centered, against the glenoid surface, the center point, or approximate center point, of the glenoid surface can be defined by the central opening of the sizer plate 240 and the channel 282 of the sizer handle 280. To create a transhumeral tunnel 1022, also referred to as a transhumeral bone tunnel, the drill bit 285 can be advanced through the humerus 1012 along an axis T, through the channel 282, and through the central opening of the sizer plate 240 until the distal end of drill enters the glenoid surface 1018, creating a pilot hole in the glenoid surface 1018. Because the drill bit 285 has a stepped configuration, as best shown in FIG. 4B, the smaller diameter can be used to initially form the transhumeral tunnel 1022 and the pilot hole, and the larger diameter can be used to increase a diameter of the transhumeral tunnel but not enter the glenoid so the pilot hole remains the smaller diameter. The pilot hole serves as a central starting hole as it can be substantially centered with respect to the glenoid surface 1018.

Alternatively, as shown in FIG. 6B, if patient anatomy allows and/or is manipulated in known manners, the drill bit 285 can be passed through the rotator interval 1020 inserted into the channel 282 to form a starter hole and/or a pilot hole in the glenoid surface 1018 at the approximate centerpoint of the glenoid defined by the central opening of the sizer plate 240, without having to pass through a transhumeral tunnel, like the tunnel 1022, formed in the humerus 1012. For example, a starter hole can be formed, with the hold being approximately in the range of about 3 millimeters to about 5 millimeters deep. This initial starting hole may not be in the optimal trajectory but it can be used to approximately centrally dock and guide the drill bit 285 when creating the pilot hole. Because the illustrated drill bit 285 is a stepped configuration, the resulting pilot hole can have multiple diameters, though in other embodiments, a single diameter drill bit may be used such that the resulting pilot hole has a single diameter. The sizing and creation of the central starting and pilot holes can be used to guide the transhumeral glenoid preparation workflow that follows. In some embodiments, the starter hole is formed as illustrated in FIG. 6B and then a pilot hole is formed, for example as described with respect to FIGS. 7A-7B below, with the starter hole being able to be used to centrally dock the glenoid guide 200 and guide the drill bit 285 when creating the pilot hole.

Following the creation of a starter and/or pilot hole in the glenoid surface 1018, a glenoid guide, such as the guide 200, can be introduced to assist in preparing the glenoid to receive an implant(s) and/or to further define the position and trajectory of the transhumeral tunnel 1022 and its entry into the glenoid surface 1018. The glenoid guide 200 can be introduced into the joint space 1010 through the rotator interval 1020. For this transhumeral glenoid preparation, an external, articulated arm positioner, such as arm positioners like a Smith & Nephew Spider arm or limb positioner, can be used, for example by attaching it to an operating room bed. Such a positioner can be used, for example, to approximately align a desired entry point on the humerus 1012 with the glenoid face or surface 1018. A surgical assistant can also be used in lieu of, or in conjunction with, the positioner.

The arm of the patient can be placed in a neutral position with the humeral resection surface 1015 facing the glenoid fossa. The foregoing notwithstanding, a person skilled in the art will appreciate that optimal positioning can depend, at least in part, on patient anatomy, the humeral resection, and/or surgeon preference, and thus other positioning may be more appropriate. It can be helpful to plan and identify the optimal trajectory for glenoid drill insertion. This may include, for example, targeting a zone that is approximately in the range of about 1 centimeter to about 2 centimeters below the plane of the humeral resection surface 1015 for a lateral entry point of the teeth 259 of the drill cannula 250. The aforementioned arm positioner and/or a surgical assistant can be used to help maintain the humerus in the desired position.

While the present disclosure discusses determining a center, or an approximate center, of the glenoid surface 1018, such as by using glenoid sizer plates (e.g., the sizer plates 240), glenoid sizer plates more generally can be used to determine a preferred location on the glenoid surface 1018, which may or may not be a center or approximate center. A preferred location may be, for example, more inferiorly positioned. A preferred location can depend, at least in part, on an anatomy of a patient, the configuration of the instruments and/or tools being used, and/or surgeon preferences, among other factors.

FIG. 7A illustrates the glenoid guide 200 coupled to the humeral resection surface 1015, and thus the humerus 1012, with a distal portion 200d of the glenoid guide 200 being disposed between the humeral resection surface 1015 and a surface of the glenoid 1018. In use, a proximal portion 200p of the glenoid guide 200 can be disposed outside of a patient's body. More particularly, as shown, the generally arcuate, or offset, arm 210 of the guide can be placed such that the distal portion 210d of the arm 210 is disposed within the rotator interval 1020 between the humeral resection surface 1015 and the glenoid surface 1018 and the proximal end 210p is disposed proximal of the humeral resection surface 1015, outside of the joint space 1010 and/or outside of the patient's body. As shown, the post 226 having the distal opening 224 formed therein can contact or otherwise abut the glenoid surface 1018 at an approximate centerpoint. The drill cannula 250 can be inserted into the opening 222 at the proximal end 210p and be advanced towards the humeral resection surface 1015 in a direction Y until the distal tip 250t of the distal portion 250d of the drill cannula 250, and thus the teeth 259, contacts the lateral cortex 1023 of the humerus 1012, for example at the aforementioned targeted zone that is approximately in the range of about 1 centimeter to about 2 centimeters below the plane of the humeral resection surface 1015. For example, the teeth 259 can be at least 10 millimeters below the resection surface. In instances in which the transhumeral tunnel 1022 have not yet been created, such as FIG. 6B, or instances in which the created transhumeral tunnel 1022 needs to have a larger diameter, the drill 285 can be advanced in the direction Y to form the desired transhumeral tunnel 1022.

As shown, the cannula-receiving opening 222 at the proximal end 210p, the drill cannula 250, and the opening at the distal end 210d are aligned along the axis LA, which is the same axis as the longitudinal axis T extending through the transhumeral tunnel 1022. Therefore, aligning the distal opening 224 with the pilot hole formed in the glenoid surface 1018 will align the opening 256 of the drill cannula 250 and the cannula-receiving opening 222 along the axis T of the transhumeral tunnel 1022.

The drill bit 285 can be passed through the drill cannula 250 and into the humerus 1012, starting from the lateral cortex 1023 until it exits the humeral resection surface 1015. A tip 285t of the drill 285 can be visualized extending beyond the post 226, prior to entering into the glenoid 1018. The bone hook 1031 (see, e.g., FIG. 7B) can help to increase visualization within the joint. This includes while the tip 285t has not yet entered the glenoid 1018. The guide 200 can be slid back, towards the humeral resection surface 1015, to better visualize the drill tip 285t. As the drill bit 285 is advanced out of the humeral resection surface 1015, it can follow the trajectory into the distal opening 224 and into the starter and/or pilot hole at the center of the glenoid surface 1018, as shown in FIG. 7B. Using the pilot hole formed in the glenoid surface 1018, the drill bit 285 can be docked therein. The glenoid guide 200, in turn, can be slid towards the glenoid surface 1018, positioned on the glenoid face, in a desired version and inclination, with the post 226 in contact with the glenoid surface 1018. A position of the humerus 1012 can be adjusted as desired to achieve a desired axis T.

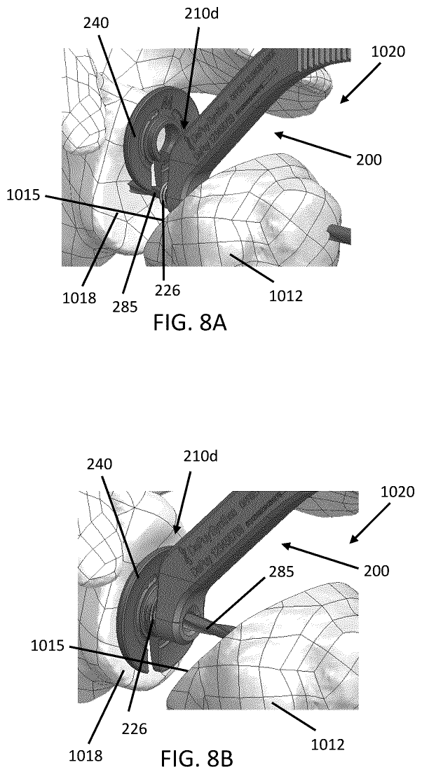

FIGS. 8A and 8B illustrate an alternative embodiment in which the sizer 240 is used to provide additional guidance and can provide enhanced tactile feedback. As shown in FIG. 8A, the sizer 240 is disposed at the surgical site. With the drill tip 285t (not visible) of the drill 285 docked in the glenoid 1018, the guide 200 can be retracted and a tool, such as various snaps tools described herein or otherwise known to those skilled in the art, can be used to slide the sizer 240 over the shaft of the drill 285, as shown in FIG. 8B. A push towards the glenoid 1018 can mate the sizer 240 to the drill 285 and can also center the guide 200, and in particular the post 226 with the sizer 240. With medial pressure, the guide 200 trajectory can be adjusted to optimize drill axis while the sizer 240 provides additional tactile feedback. The humerus position can also be adjusted as necessary to achieve optimal trajectory.

Once that desired alignment is achieved, the drill bit 285 can be advanced further in the direction Y until bicortical penetration is achieved. For example, the drill bit 285 can be advanced down the glenoid fault at least about 25 millimeters. It can be confirmed that the driver 285 exits the scapula as desired. Subsequently, the drill bit 285 can be removed from glenoid surface 1018 and the guide 200, thus leaving the surgical site, and the glenoid guide 200 also can be removed from the surgical site to provide additional space for further glenoid preparation steps. These components can be existed through the rotator interval 1020, for example. Optionally, after the glenoid guide 200 has been evacuated from the surgical site, the drill bit 285, or another elongate structure, can be inserted through the tunnel 1022 in the direction Y and into the face of the glenoid 1018 for clear visualization to confirm placement. In some embodiments, the drill bit 285 or another component can be inserted through the drill cannula 250, and the transhumeral tunnel 1022, to serve as a guidewire during the procedure.

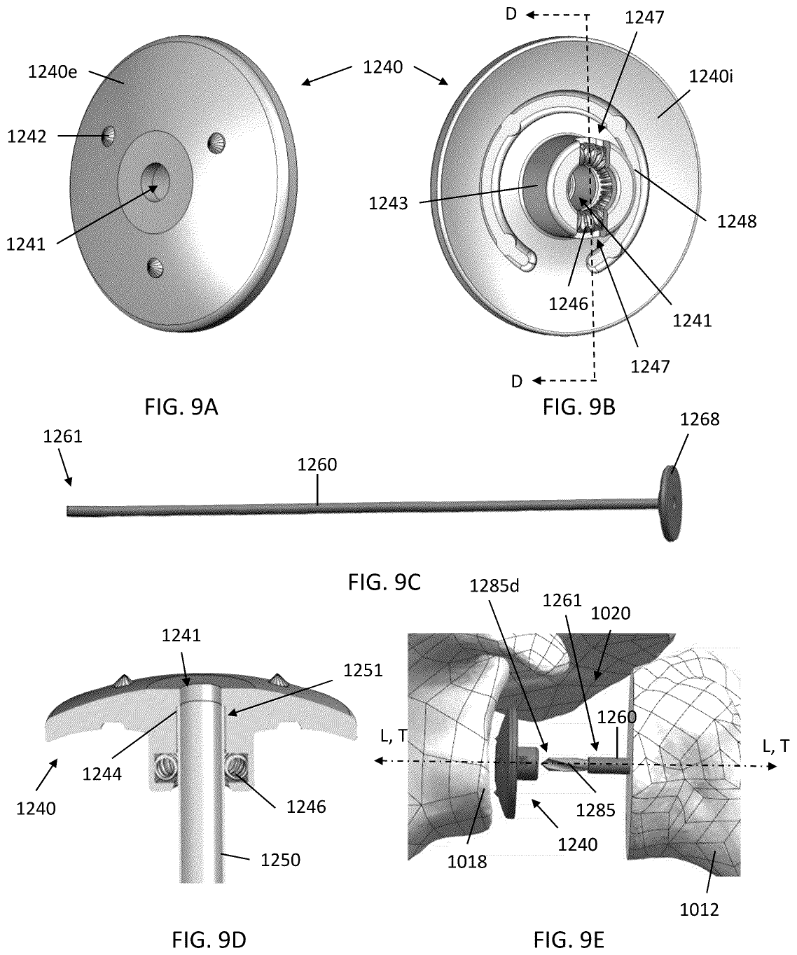

FIGS. 9A-9E illustrate an alternative form of using a sizer plate or the like to determine a size and/or other related parameters for an implant(s) to be received by the glenoid and determine a center or approximate center of the glenoid. More particularly, FIGS. 9A-9B and 9D-9E illustrate an embodiment of a low profile glenoid plate or sizer 1240 designed to be engaged with a cannulated drill guide 1260, illustrated in FIG. 9C, and configured to locate and mark the center of the glenoid surface 1018. The glenoid sizer 1240 illustrated is a substantially circular disc with a convex external surface 1240e configured to abut against the glenoid surface 1018, an opposed concave internal surface 1240i, and a central opening 1241 extending therebetween. Glenoid plates or sizers, such as the sizer 1240, can be manufactured in various diameters to match patient anatomy. Various sizer plates, such as for sizing the humeral resection surface 1015 and/or a glenoid as provided for herein or otherwise known to those skilled in the art can be used to select the appropriately sized glenoid sizer for the patient. Alternatively, or additionally, multiple guides or sizers 1240, similar to the sizers 240, can be used to determine the appropriate sized sizer 1240 be used with the patient. When the convex surface 1240e abuts against the surface of the glenoid 1018 with a perimeter of the sizer 1240 laying within and proximal to a perimeter of the glenoid surface edge, the central opening 1241 of the guide substantially aligns with and marks a center point of the face of the glenoid 1018.

A plurality of protrusions 1242 can be formed on the convex external surface 1240c. In the illustrated embodiment, three protrusions 1242 are formed, the protrusions 1242 being radially and equidistantly spaced from each other and from the central opening 1241. Other numbers of protrusions, including fewer or greater than three, can be used, as can other configurations. The protrusions 1242 can help stabilize the sizer 1240 against the surface of the glenoid 1018.

The internal surface 1240i can include a central mount 1243 that is substantially circular and helps define the central opening 1241. As shown, the central mount 1243 extends away from the internal surface 1240i, and away from the external surface 1240e. The mount 1243 can be configured to receive an instrument, as shown the cannulated drill guide 1260 in FIG. 9C, therein such that the instrument can be mated to the glenoid sizer 1240. The cannulated drill guide 1260 can include a distal end 1261 configured to mate with the sizer 1240 and/or other components used in a surgical procedure and a proximal end 1268 having a flange disposed therein to assist a user in rotating or otherwise manipulating the drill guide 1260. In some embodiments, a portion of the volume formed between the inner and external surfaces 1240i and 1240e can be removed such that openings are formed through the sizer 1240. Such openings can, for example, increase visualization to aid the user in seeing the bone while placing the component.

With reference to FIG. 9D, an inner surface 1244 formed by an inner wall that defines central opening 1241 can be configured to engage the distal end 1261 of the cannulated drill guide 1260. A spring 1246 disposed in the central mount 1243 can provide biasing that helps the inner surface 1244 engage and hold the cannulated drill guide 1260 within the mount 1243. In at least some embodiments, the drill guide 1260 can slide into the central opening 1241 and form a slip fit with the inner surface 1244 of the mount 1243, with the spring 1246 acting to provide a frictional drag fit to help secure the sizer 1240 onto the cannulated drill guide 1260 during manipulation in the joint. One or more slots, as shown in FIG. 9B two opposed slots 1247, can be formed in the mount 1243 to aid inserting the spring 1246 at its illustrated location with respect to the mount 1243, and to provide cleaning and visualization as needed. In at least some embodiments, a radial groove or channel 1248 can be formed in the internal surface 1240i. As shown, the radial groove 1248 extends circumferentially around a majority, but not an entire, circumference of the sizer 1240 such that it forms a C-shape. The groove 1248 can represent a peripheral ring that can be part of a glenoid implant and can provide surgeons a reference as to where it will be while positioning the sizer 1240 and associated drill (e.g., a drill 1285, as shown in FIG. 9E) before reaming.

FIG. 9D illustrates one way by which the glenoid sizer 1240 can be inserted into the joint space through the rotator interval 1020. The convex surface 1240e of the sizer 1240 can be aligned with the surface of the glenoid 1018 such that the central opening 1241 aligns with the center of the glenoid 1018. The drill guide 1260 can be passed through a drill cannula (e.g., the drill cannula 250), within the transhumeral bone tunnel until the distal end 1261 of the drill guide 1260 is inserted into the mount 1243 and secured to the sizer 1240. The drill guide 1260 can position the sizer 1240 such that it is proximate to or in contact with the surface of the glenoid 1018 such that the sizer 1240 can be used to help mark the center of the surface of the glenoid 1018. More particularly, the central opening 1241 marks the center of the surface of the glenoid 1018 so that in instances when a guide pin, such as a guide pin, drill, or drill bit 1285 illustrated in FIG. 9E or a guidewire is used, it can be passed through a drill cannula (e.g., the drill cannula 250) and the drill guide 1260, following the axis T, which is colinear with the longitudinal axis L, until the distal end penetrates the center of the glenoid 1018. After fixing a distal end 1285d of the drill 1285 in the center of the glenoid 1018, the glenoid sizer 1240 can be disengaged from the cannulated drill guide 1260, for example by an axial pull, and removed from the surgical site. In other embodiments, the drill of FIG. 4B can be used.

### Bullet Driver(s) Reaming

FIGS. 10A-10E illustrate the use of the glenoid reamer attachment 700 and the driver 290 of FIGS. 5A-5E. A size of the reamer attachment 700 can be selected based on the size of the glenoid sizer 240, or other sizes disclosed herein or otherwise known to those skilled in the art. More particularly, with the glenoid sizer 240 being determined to be the appropriate size for the glenoid of the patient, which in turn informs the appropriate size of the prosthesis or implant that will engage or otherwise be used with the glenoid 1018, the glenoid sizer 240 also informs the selection of the size of the reamer attachment 700 as the selected reamer attachment 700 will influence the size, shape, speed, and/or other parameters associated with modifying the surface of the glenoid 1018 in the desired manner for the surgical procedure.

Prior to inserting the reamer attachment 700 to the surgical site, the arm cam be positioned in adduction with slight external rotation. This can aid in assembly of components during the procedure. Use of a bone hook(s), like the hook 1031, can also help increase visualization within the joint space 1010. The reamer attachment 700 can be introduced into the glenohumeral joint space as shown in FIG. 10A, through the rotator interval 1020, such as by passing it above or below the subscapularis. A surgical tool, such as snaps (see, e.g., a tool 800 illustrated in FIG. 10E), can be used to hold the reamer attachment 700 and navigate the attachment to this location.

The driver 290 can be separately introduced into the glenohumeral joint space for alignment with the reamer attachment 700. The driver 290 can be designed to follow the axis LB, as well as the longitudinal axis T extending through the transhumeral bone tunnel formed in the humerus 1012, created by the drill 285. In at least some embodiments, the driver 290 can be attached to a quick connect driver handle or paddle handle for case of manipulation. The driver 290 can be pushed through the transhumeral bone tunnel formed in the humerus 1012, and the tip 294 and distal end 290d of the driver 290 into the glenohumeral joint space by providing a force in the direction J. More particularly, the central longitudinal axis LB extending through the central opening 732 of the reamer attachment 700 can be aligned with the central longitudinal axis LA that extends through the driver 290 and then the driver 290 can be advanced in the direction J, along the longitudinal axis LA, and thus along the longitudinal axes LB and T. The advancement of the driver 290 in the direction J can be done with a slight twisting motion to align the hex-shaped portion 296 of the driver 290 with the hex-shaped portion 734 of the reamer attachment 700, as better illustrated by FIGS. 5C-5E. A further push in the direction J can engage and mate the reamer attachment 700 with the driver 290, which can be felt by the surgeon via tactile feedback when one mates to the other. Further, visual confirmation of the engagement can be seen as the tip 294 protrudes out past the distal-most portion of the reamer attachment 700, i.e., the pilot blades 720, as shown in FIG. 10B. More particularly, as shown, the driver 290 passes into and through the central opening 732 formed through the base 710, the reaming blades 740, the central tunnel 730, and the pilot blades 720, with the tip 294 extending distally beyond the pilot blades 720, and is disposed proximate to the glenoid 1018. Further, in at least some embodiments, a marking 295, as shown a laser marking, can be formed on the shaft of the driver 290 at a location that indicates to a user that the reamer attachment 700 is mated to the shaft 290. As a result, tactile confirmation and at least two visual confirmations of the attachment 700 being secured to the shaft 290 are provided by the illustrated design. During the course of the procedure, it can be helpful to maintain rotation during reaming into and out of the bone to ensure that the reamer attachment 700 and the driver 290 do not disengage, at least because they are assembled with a friction fit.

Subsequently, the rounded distal tip 294 of the driver 290 can be inserted into the pilot hole formed in the surface of the glenoid 1018, moving the driver 290, and thus the reamer attachment 700, in the direction J, towards the glenoid 1018. The action of inserting the rounded distal tip 294 into the pilot hole can be performed without the assistance of a guidewire or other components for guidance which can be beneficial in at least some instances because not using a guidewire provides for one less component, and sometimes fewer steps, to be used and/or engage the surface of the glenoid 1018, among other benefits understood by a person skilled in the art for not using a guidewire. Further, procedures that do not require the use of a guidewire can enhance the usability of the devices and techniques in a confined space, and can also make it easier to attach a reamer to a shaft because it prevents the need of having to go over a guidewire and then onto a shaft guided by that guidewire to properly situate the reamer. If the arm of the patient has moved, the aforementioned arm positioner can be used to align the arm such that the axes LA, LB, and T are colinear, as shown in FIG. 10B. As designed, the pre-drilled pilot hole can guide the trajectory of the driver 290 during reaming.

With reference to FIG. 10C, once the desired orientation between the reamer attachment 700 and the surface of the glenoid 1018 is achieved, the driver 290 can be operated to ream the glenoid 1018. This can be done by hand, with a driver handle for example, and/or it can be done under power, such as using modified trinkle connection like the previously referenced trinkle connection or other such connections known to those skilled in the art. To the extent power is used, generally a “ream” setting should be selected and the reamer attachment 700 typically should be actuated prior to contacting the glenoid surface 1018. The driver 290 and reamer attachment 700 can continue to be operated, moving them in the direction J as shown, until the desired depth is reached and/or until the face of the reamer, defined by the reamer face 710f, (not visible; see FIG. 5B) is flush with a face of the glenoid 1018, as shown in FIG. 10D. If excess force appears to be required to perform the reaming, it may be an indication that the driver 290 is off-axis from the pilot hole. If this occurs, it can be helpful to remove the reamer attachment 700 from bone and realign the driver 290 with the pilot hole.

Turning to FIG. 10E, after completion of glenoid reaming, the reamer attachment 700 and related components can be removed from the surgical site. This can be done, for example, under power, which can minimize a risk of premature disconnection from the driver 290. In some instances, to enhance visualization, a retractor(s) (not shown) can be placed on the humeral resection surface 1015 for protection. The driver 290 can be detached from the reamer attachment 700, for example by pulling back on the driver 290 in a direction K until the reamer attachment 700 contacts an object to disengage the reamer attachment 700 from the driver 290. For example, in the illustrated embodiment, a snaps tool 800 is provided loosely over the driver 290 and as the driver 290 is pulled in the direction K, the reamer attachment 700 can contact the snaps tool 800 to become disengaged from the driver 290. The snaps tool 800 can also protect the humeral resection surface 1015. The snaps tool 800 can enter the surgical site using techniques provided for herein or otherwise known to those skilled in the art, including entering through the rotator interval 1020. In some embodiments, pulling the driver 290 in the direction K can cause the reamer attachment 700 to contact a retractor(s) and/or the humeral resection surface 1015 to cause disengagement. The reamer attachment 700 can be removed from the surgical site, for example through the rotator interval 1020, using a surgical tool like the snaps 800.

The driver 290 can also be vacated from the surgical site, or alternatively, one or more other tools can be introduced into the glenohumeral joint space in a manner similar to the way the reamer attachment 700 was introduced. The instruments can provide other preparations to the glenoid as desired. Operation of such other tools can occur similar to operation of the reamer attachment 700, though a person skilled in the art will appreciate variations that may be appropriate in view of the purpose and operational parameters of the tool being used. Accordingly, the present disclosure provides for the ability for different tools, including but not limited to variously shaped and configured reamer attachments, as well as other tools for treating a glenoid, to be separately disposed in the glenohumeral joint space, coupled to the driver 290 or other similar shaft, operated, disconnected from the driver 290 or other similar shaft, and then removed from the surgical site. Some non-limiting examples of such tools include a Cobb Elevator surgical tool, which can be used, for example, to remove cartilage before reaming, and/or electrocautery devices, which can be used, for example, to remove soft tissue structures from bone, as well as to mark positions and/or centering location(s) in conjunction with performing a surgical procedure.

Reaming with a Guidewire

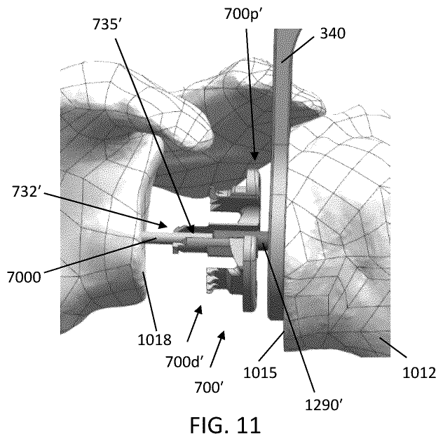

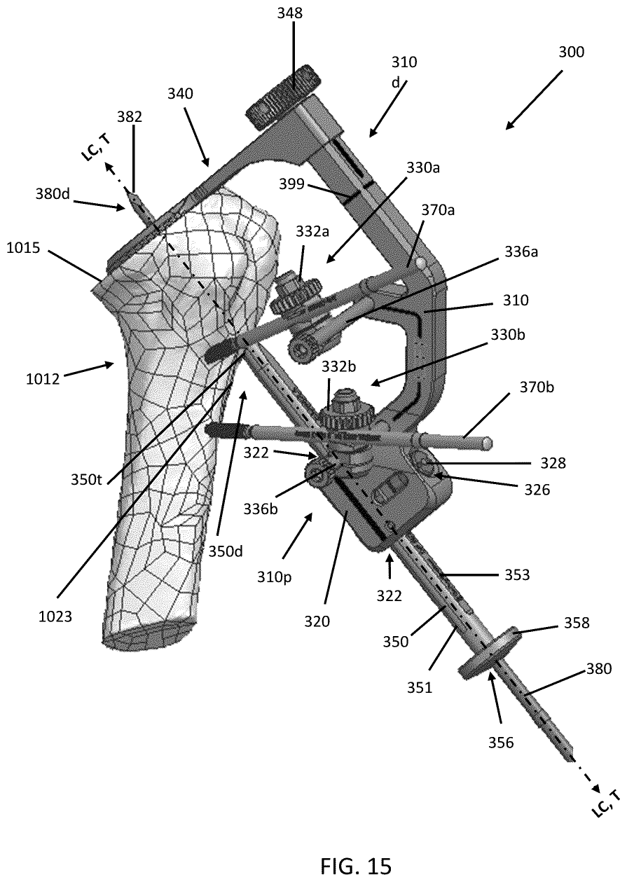

Alternative to reaming using a driver, a guidewire can be left in place in the joint space to further guide a reamer attachment or reamer, like the reamer attachment 700, along the axis T and into the glenoid surface. It can be helpful, in at least some instances, to ream with a guidewire in place to provide additional guidance. A configuration that employs a guidewire 7000 is illustrated in FIG. 11, with an alternative reamer attachment 700′ being used in conjunction with the same. Alternatively, a drill, like the drills 285 or 1285, can be used a guidewire. In the illustrated embodiment, the guidewire 7000 is used in conjunction with, for example, a humeral sizer attachment 340 coupled to a humeral guide 300 (not shown), as illustrated and described below with respect to FIGS. 15-17, though in other embodiments, the guidewire 7000 can be used without a humeral guide 300.

In some embodiments, after the drill 1285 is removed following the actions described above with respect to FIG. 9E, the guidewire 7000 is introduced. This can be done, for example, by passing it through the drill cannula 250, through the transhumeral tunnel formed in the humerus 1012, through the glenohumeral joint space, and into the glenoid 1018 at the location of the aforementioned pilot hole such that it extends along the colinear longitudinal axes L and T. In the embodiment of FIG. 11, the guidewire 7000 is passed through a cannulated bullet or drill cannula 350 associated with a humeral guide 300 (see FIG. 15), through the transhumeral tunnel formed in the humerus 1012, through the glenohumeral joint space, and into the glenoid 1018 at the location of the aforementioned pilot hole such that it extends along the colinear longitudinal axes L and T. The preplaced guidewire 7000 provides a reliable trajectory along the axes L and T for the reamer attachment 700′ to follow such that when it meets the surface of the glenoid 1018, the plane of the attachment 700′ is substantially parallel and center to a plane defined by a surface of the glenoid 1018. The guidewire 7000 can be, for example, a metal guidewire having a diameter of approximately 2.5 millimeters such that other devices and/or components used during the surgical procedure can pass over it to maintain alignment.

The alternative reamer attachment 700′ can be akin to the reamer 700 except that a radial slot 735′ can be formed therein, the slot 735′ extending from a proximal end 700p′ of the reamer attachment 700′ to a distal end 700d′ of the reamer attachment 700′. The radial slot 735′ provides access to a central opening 732′ formed in the reamer attachment 700′ such that the guidewire 7000 can be passed through the slot 735′ as the reamer attachment 700′ is associated with the guidewire 7000. In this configuration, the reamer attachment 700′ can be operated while being guided by the guidewire 7000. As shown, the reamer attachment 700′ can be operated by way of a driver 1290′ coupled thereto in a manner similar as the reamer attachment 700 and the driver 290. The driver 1290′ can be disposed around the guidewire 7000 such that the guidewire 7000 is disposed through a cannulated shaft of the driver 1290′ and the reamer attachment 700′ is disposed radially around the guidewire 7000. The central opening 732′ of the reamer attachment 700′ can be configured in a manner that allows the guidewire 7000 to be passed therein via the slot 735′ while allowing the reamer attachment 700′ to slide along the guidewire 7000, such as by way of the driver 1290′, without become disassociated from it. In at least some embodiments, the driver 1290′ can have one or more distal slot features (not shown) formed therein and configured to mate with complementary pin protrusion features (not shown) located with the central opening 732′ of the reamer attachment 700′.

Operation of the reamer attachment 700′ can be similar to the operation described above with respect to the reamer attachment 700, with a person skilled in the art understanding how to operate the reamer attachment 700′ while a guidewire is associated therewith and thus understanding differences between operation of the two attachments 700 and 700′ without having to explicitly describe them herein. For example, the cannulated drill guide 1290′ can be configured to be used in conjunction with the reamer attachment 700′, coupling together using techniques provided for herein or otherwise known to those skilled in the art, including but not limited by forming a snap fit between the two components. The cannulated drill guide 1290′ can snap into the reamer attachment 700′ such that it provides sufficient rotational and axial force to the reamer attachment 700′. The cannulated drill guide 1290′ can pass along the guidewire 7000 in operation of the reamer attachment 700′. A person skilled in the art will appreciate the cannulated drill guide 1290′ could be used in conjunction with a non-guidewire configuration, although without a guidance mechanism, difficulties in providing proper centering with respect to the desired position on the glenoid 1018 may occur. Further, to the extent the present disclosures do not explicitly provide for it, a person skilled in the art will understand how to disassociate components like the glenoid guide 200, or the humeral guide 300, from a guidewire 7000 in view of the present disclosures and the applicability of one connection technique to the various components that are used together herein.

Notably, in this disclosure, various shafts are used to operate reamer attachments, including but not limited to a cannulated drill guide 1290′, and a driver 290. More generally these types of components can be described as shafts, drive shafts, or drivers, with the present disclosure enabling a surgeon to couple such shafts to a reamer attachment, glenoid guide, and/or other tool(s) within the glenohumeral joint space to subsequently operate and/or otherwise use the reamer attachment, glenoid guide, and/or other tool(s).

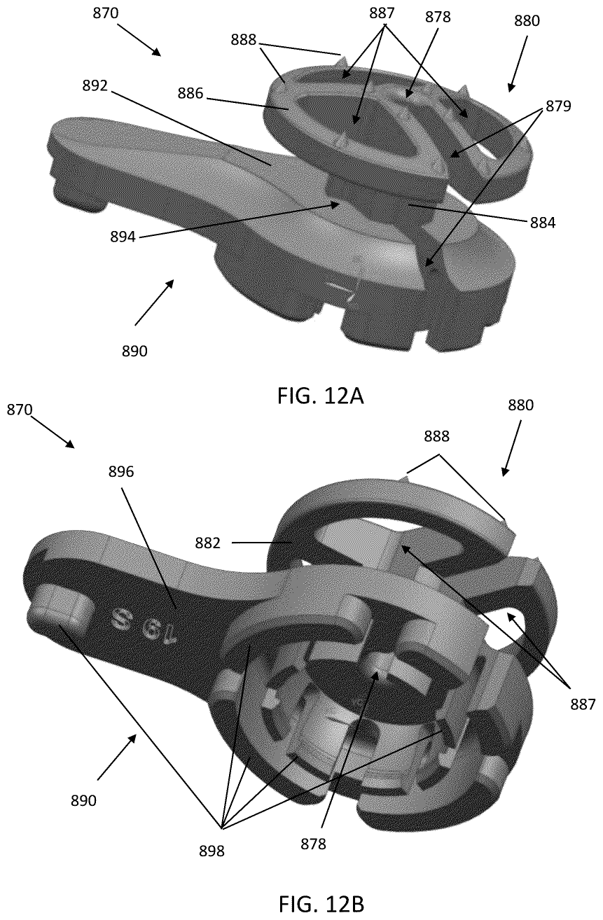

FIGS. 12A-12B illustrate one example of a guidewire adapter 870 that can be used in conjunction with introducing a guidewire, like the guidewire 7000, to the surgical site. The guidewire adapter 870 can be configured to be coupled to a sizer plate, like a sizer plate 172 (see FIG. 16D) associated with a humeral sizer attachment, like the humeral sizer attachment 340 (see FIGS. 15-17). As shown, the guidewire adapter 870 includes an upper portion 880 and a lower portion 890. The adapter 870 can include a central opening 878 that extends through both the upper and lower portions 880 and 890. When coupled to a sizer plate, like the sizer plate 172, the adapter 870 can be configured so that the central opening 878 is aligned with a central opening (see central opening 178 of FIG. 16D) of the sizer plate (see sizer plate 172 of FIG. 16D). As a result, a central opening (e.g., the central opening 178) of the sizer plate (e.g., sizer plate 172) can be centered with respect to the humeral resection surface 1015, and the longitudinal axes L and T, described above, can pass through the central opening (e.g., the central opening 178) of the sizer plate (e.g., sizer plate 172) and the central opening 878. Accordingly, a guidewire, such as the guidewire 7000 (not shown) can be passed through the central opening (e.g., the central opening 178) of the sizer plate (e.g., sizer plate 172) and the central opening 878.

The upper portion 880 can be substantially circular, although other shapes and sizes are possible. A shape and size (e.g., diameter) of the upper portion 880, as well as the lower portion 890 and the adapter 870 more generally, can be based, at least in part, on the anatomy of the patient, and thus a shape and size of the sizer plate can drive the shape and size of the guidewire adapter 870 and its upper and lower portions 880 and 890. An interior face 882 of the upper portion 880 adapter can include a male coupling component 884 configured to couple to a complementary female coupling component 894 of the lower portion 890. This can provide for a snap-fit between the upper and lower portions 880 and 890. A person skilled in the art will appreciate a variety of coupling configurations that can be used to mate the two portions 880 and 890′. An exterior face 886 of the upper portion 880 can have a plurality of spikes or spike features 888 formed thereon, which can be used to help maintain a position of the guidewire adapter 870 at the surgical site, for instance by engaging bone at or proximate to the surgical site. A plurality of openings 887, as shown three, can be formed in the upper portion 880 to provide visualization and/or paths for cut tissue, fluids, debris, and other materials to pass through during operation of attachments, although in some embodiments the adapter can be removed prior to operation of such attachments, such as prior to reaming.

The lower portion 890 can have a relatively smooth interior face 892 that includes the female coupling component 894. The exterior face 896 of the lower portion 890 can have collet-type configuration with a plurality of location features 898 formed thereon. The location features 898 can be complementary to openings formed in the sizer plate (e.g., the sizer plate 172), thus allowing the guidewire adapter 870 to be securely coupled, e.g., snap-fit, onto the sizer plate (e.g., the sizer plate 172) as desired during placement of a guidewire like the guidewire 7000.

A slot 879 can extend through both the upper and lower potions 880 and 890, similar to the central opening 878. The slot 879 can allow for the adapter 870 to be removed from the surgical site after the guidewire has been placed but prior to performing actions like glenoid reaming.

Implanting a Glenoid Implant(s) and/or Prosthesis(es)

Prior to inserting a glenoid implant and/or prosthesis, trialing and/or sizing can be performed. This can be done, for example, using the INHANCE™ surgical technique (from Johnson & Johnson of New Brunswick, NJ), as disclosed at https://www.jnjmedtech.com/en-US/pdf/inhancetm-shoulder-system-anatomic-surgical-technique, the content of which is incorporated by reference herein in its entirety. While performing the trialing, which can involve placing one or more trial or trial implants at the surgical site to determine desired fit and configuration, confirmation should be made that the trial has acceptable backside support, for instance by visually confirming the same via a window(s) of the trial being used. In at least some instances, an impactor tip (see, e.g., the tip 206) can be used to seat the trial(s) during trailing. After the appropriate prosthesis, sometimes referred to as an implant, is determined, steps can be taken to introduce and implant it into the glenoid 1018.

As shown in FIGS. 13A-13B, a glenoid guide 200′ can be introduced into the surgical site to implant a prosthesis, as shown a glenoid implant 204, into the glenoid surface 1018. In at least some embodiments, the glenoid guide 200′ can instead be the glenoid guide 200. The glenoid guide 200′ is similar to the glenoid guide 200, and thus additional description of the same is not needed. As shown, it includes an offset arm 210′ having a proximal end 210p′ with an opening 222′ and a distal portion end 210d′ with an opening 224′, with the proximal end 210p′ having a flat surface 212′ and the distal end 210d′ being configured to receive a component, as shown the implant 204. The distal end 210d′ can include, for example, a post 226′ (not visible) akin to the post 226. The implant 204 can have a concave surface 204c, configured to mimic a surface of the glenoid 1018, the implant 204 being configured to receive a complementary humeral head prosthesis that is implanted at the humeral resection surface 1015. Implants of the nature of the implant 204 are known to those skilled in the art, and thus further disclosure about the glenoid implant are not necessary.

The distal end 210d′ of the glenoid guide 200′, and more particularly the post 226′ (not visible) can be configured to couple to the implant 204. The implant 204 can be securely mated to the distal end 210d′ of the arm 210′ of the guide 200′ using any techniques known to those skilled in the art for coupling two components together, such as a threaded connection created by the threaded post 226′, a snap-fit connection, male-female engagement mechanisms, etc. Further, in at least some embodiments, a glenoid-facing surface 204g of the implant 204 can be treated with cement per the INHANCE™ surgical technique (from Johnson & Johnson of New Brunswick, NJ), details about the technique being available at the link previously provided above, to help better secure the implant with respect to the glenoid 1018.

The implant 204 can be removed from sterile packaging, such as a thermoformed tray and retainer lid. As packaged, the implant can be packaged with an inserter tip. To allow for a touch-free introduction of the implant, the retainer lid can be removed and the post 226′ of the glenoid guide 200′ can be threaded into the inserter tip of the implant 204, as described above. The implant 204 can be placed into the joint space in a manner such that there is axial alignment between a center axis I of the implant 204 and the longitudinal axes LA, LB, and T, as shown in FIG. 13B. As shown in FIG. 13A, the implant 204 can be situated with respect to the glenoid guide 200′ such that the axis I extends centrally through the openings 222′ and 224′. Once the positioning of the implant 204 is at the desired location at the surgical site, the glenoid guide 200′ can be used to provide an impaction force to the implant 204 to secure the implant with respect to the glenoid 1018. More particularly, a force in a direction P can be applied to the flat surface 212′ located on the proximal end 210p′ of the arm 210′, for instance using a hammer or mallet. Application of the force in the direction P can help to drive the implant 204 into the glenoid 1018 for subsequent seating with respect to the glenoid 1018. The implant 204 can be separated from the guide 200′ using known techniques, including unthreading one from the other and/or sliding the post 226′ (not visible) off the implant 204. Other implants that can be inserted in similar manners include the prosthesis 24 of FIG. 1A and the prosthesis 60 of FIG. 1B.

In at least some embodiments, as shown in FIG. 14A, an impactor tool or tip 206 can be coupled to the post 226′ (not visible) of the distal end 210d′ of the arm 210′ of the glenoid guide 200′. In the illustrated embodiment, the impactor tool 206 includes a glenoid-facing surface 206g that is convex and substantially complimentary to the concave surface 204c of the implant 204. In use, as shown in FIG. 14B, the glenoid guide can have a force in the direction P applied to the flat surface 212′, for instance by a hammer or mallet, to help drive the impactor tool 206 into the implant 204 to further seat the implant 204 at the desired location. Use of the impactor tool 206 can help minimize any damage or trauma to the concave surface 204c because the impactor tool 206 can be made of a material that does not typically cause damage (e.g., scratches, dents, etc.) to the concave surface 204c of the implant 204.

A geometry of the implant 204 is typically aligned with the complementary geometry formed in the surface of the glenoid 1018, the complementary geometry formed in the surface of the glenoid 1018 being primarily formed by the reamer attachment 700. In at least some embodiments, as the implant is maneuvered to enter the recesses formed in the surface of the glenoid 1018, it can seat approximately half-way into the surface of the glenoid 1018, though in other instances it can be more or less flush with respect to the surface of the glenoid 1018 than that. In some instances, bone cement can be used within recesses formed in the surface of the glenoid 1018 to further fix the implant within the glenoid 1018.

This resulting configuration of the implant placement can be articulated and assessed to ensure the sizing and configuration is appropriate. The implant can be removed and replaced to obtain desired joint tension. While the illustrated embodiment illustrates the glenoid guide 200′ being used for disposing the implant 204 in the glenoid 1018 without a guidewire, a person skilled in the art, in view of the present disclosures, will understand how the illustrated technique can be adapted for use with a guide wire that passes through the transhumeral tunnel 1022, through the openings 222′ and 224′ of the glenoid guide 200′, and into the pilot hole formed in the surface of the glenoid 1018.

### Humeral Guide(s)

FIG. 15 illustrates an embodiment of a surgical device, referred to herein as a humeral guide 300, also referred to as a surgical guide, a surgical guide frame, or a humeral guide frame. The guide 300 can be used in various surgical procedures and/or for various purposes, but in at least some instances it can be used to maintain positional guidance during various steps in a tissue sparing shoulder arthroplasty procedure in which the subscapularis remains attached to the scapula and proximal humerus. Details about various configurations of a humeral guide are disclosed in U.S. Provisional Patent Application No. 63/579,952, entitled “Humeral Surgical Guides, Instruments, and Techniques for Use in Tissue Sparing Shoulder Arthroplasties,” filed Aug. 31, 2023, as well as a U.S. non-provisional patent application entitled “Humeral Surgical Guides, Instruments, and Techniques for Use in Tissue Sparing Shoulder Arthroplasties,” filed on an even date herewith, the content of each which is incorporated by reference herein in its entirety. Because details of those guides are disclosed in these other applications, only a high-level overview of features relevant for purposes of using the guide 300 to identify a center of a glenoid are provided for herein.

The humeral guide 300 can include a rigid arm 310 with a carriage or hub 320 coupled to and/or disposed on a proximal portion 310p of the arm 310. The rigid arm 310 can be sized and shaped to allow for proper centering and alignment between, for example, a cannulated bullet or drill cannula 350, also referred to as a bullet or a cannula, coupled to or otherwise associated with the hub 320 and one of various modular attachments that can be used in conjunction with the guide 300, such as a humeral sizer attachment 340, also referred to more generally as a sizer attachment. As shown, the humeral sizer attachment 340 is coupled to a distal portion 310d of the arm 310.

The drill cannula 350 can be an elongate, substantially cylindrical or tubular shaft having a base or handle 358 located at a proximal end 350p, a distal end 350d having a distal tip 350t, and an intermediate portion or length extending therebetween. A planar contact surface 353 can be disposed on an outer surface of the intermediate portion of the drill cannula 350 to provide a surface for a locking mechanism, such as a spring-loaded release button 328 disposed in an opening 326 formed in the hub 320 that is biased towards a cannula-receiving opening 322, to contact and lock the position of the drill cannula 350. In some embodiments, ratcheting teeth 351 can be disposed on an outer surface of the intermediate portion of the drill cannula 350 or along the planar contact surface 353, which can be engaged by a locking tooth associated with the spring-loaded release button 328. The drill cannula 350 further includes an opening 356 extending through an entirety of a length of the drill cannula 350. The opening 356 can be sized and shaped to allow a drilling component, such as a drill pin or guide pin 380, to pass through the drill cannula 350 and into the bone in which a bone tunnel is to be formed such that the distal end 380d of the drill exits substantially center and substantially perpendicular to the humeral resection surface 1015 as shown in FIG. 15. The distal end 350d of the drill cannula 350 can be tapered, for instance by varying a thickness of a wall of the drill cannula 350, to make it easier to push the drill cannula 350 through soft tissue and against the surface of the humerus. In some embodiments, bone engaging features, such as teeth (not visible because obscured by a bone pin 370a), can be formed at the distal tip 350t. The teeth can help stabilize the location of the distal end 350t of the drill cannula 350 with respect to the bone. Further a distal most end of the distal portion 350d of the drill cannula 350 can be angled to match the angle of the humerus 1012. The stabilization provided by features such as the teeth and angled distal end 350d can set a more accurate trajectory for the bone tunnel. The cannula-receiving opening 322, along with the drill cannula 350, sets the location and trajectory of a bone tunnel or bore to be drilled through the humerus 1012 from the lateral cortex 1023 to the humeral resection surface 1015, the tunnel substantially center and substantially perpendicular the humeral resection surface 1015. The path is illustrated by a longitudinal axis LC extending through the cannula-receiving opening 322, in turn aligning with a longitudinal axis T of a transhumeral bone tunnel. The terms tunnel and bore will be used interchangeably throughout the specification as it pertains to forming a hole in a bone. In the illustrated embodiment, the formed transhumeral bone tunnel is at a different location with respect to the humerus 1012 than the transhumeral bone tunnel described with respect to FIGS. 6A and 7A-7B, with the start of the transhumeral bone tunnel of FIG. 15 being illustrated further down, or more distally down, the lateral cortex 1023.

The guide 300 includes one or more fixation features 330a, 330b, also referred to as bone pin-receiving fixation features, among other names, that are configured to position bone pins 370a, 370b received therein at desired locations with respect to the humerus 1012, and thus the surgical site. The fixation features 330a, 330b, which can be, for example, bone pin clamps 332a, 332b, along with support rods 336a, 336b coupling the bone pin clamps 332a, 332b to the arm 310, among other components provided for herein, can be considered part of and/or used in conjunction with the guide 300. Use of such fixation features 330a, 330b during identification of identifying a center of a glenoid is optional.

### Humeral Sizer Attachment(s)

FIGS. 16A-16C further illustrate the humeral sizer attachment 340 that can also be used as an attachment to the guide 300, and also referenced earlier with respect to some non-limited embodiments of using a guidewire (see, e.g., the guidewire 7000 of FIG. 11). The attachment 340 can include a substantially flat, rigid base 342 having an arm portion 342a and an alignment and sizing portion 342b, also referred to as a plate portion. As shown, the arm portion 342a can extend from a proximal-most end 342p, the location at which the attachment 340 can be coupled to the rigid arm 310, and terminates where a circular shape that primarily constitutes the plate portion 342b begins. In the illustrated embodiment, the plate portion 342b comprises a circular body 341 formed at a distal end 342d of the base 342, the circular body 341 having a plurality of openings 343a, 343b formed therein. As shown in FIG. 16B, there are four outer openings 343b, each similarly sized and shaped and disposed radially around a central opening 343a. The rigid base 342 has a length from a proximal-most end illustrated at a proximal end 342p to a central opening 343a such that when the humeral sizer attachment 340 is mated with the rigid arm 310, the central opening 343a is on the same axis LC as the cannula-receiving opening 322. Further, a plane PL defined by the plate portion 342b of the attachment 340 can be orthogonal or substantially orthogonal to the longitudinal axis LC. As shown, the plane PL extends through a primary surface of the sizing portion 342b of the attachment 340, such as an entirety of the top surface.

The central opening 343a can be centrally located with respect to the circular body 341 and can be configured to receive a drill bit or guide pin, such as the drill bit 380 (not shown in FIG. 16A, but see FIG. 15), passed through the drill cannula 350. The central opening 343a can be used, for example, to provide alignment of other portions of the guide 300, or components with which the guide 300 is used such as the drill cannula 350 and drill bit 380. More particularly, the central opening 343a can be used, for example, to locate and/or mark an approximate center point of the humeral resection surface 1015, as shown in FIGS. 16B and 16C, as well as of the glenoid 1018, as detailed thereafter.

A window 344 can be formed in the distal end of the arm portion, proximal to where the arm portion 342a joins with the plate portion 342b. A user can align an anatomical landmark, such as the bicipital groove, within the window 344. The arm portion can further include markings relating to various measurements of the humeral resection surface 1015 or an appropriately sized implant. Referencing the humeral resection surface 1015 like as shown in FIG. 16B allows a user to pick a nominal sizer and reference a marking or indicator 345 on the arm portion 342a. Accordingly, when the sizer attachment 340 is inserted into the joint space as shown in FIG. 16C, the user can laterally line-up a line 1011 formed on the bicipital groove within the window 344, and push the sizer attachment 340 into the joint until that reference marking 345 is reached, thus setting a medial lateral positioning on the humeral resection surface 1015. Holding that positon, the user can manually center, for example by palpation or visual reference, the anterior to posterior centering using the bone and the plate portion 342b. The provided locators centralize the sizer attachment 340 to what a user may want and can, if desired, ignore most inferior position where they may nibble bone off inferiorly in the end. This locating process can set the definitive implant to be located properly within rotator cuff muscles.

The sizer attachment 340 can be used to both define a center, or a central location, of the humeral resection surface 1015, or other desired location to receive an implant, as well as to size the humeral resection surface 1015 for determination of the size of the implant and/or prosthesis to be used at the surgical site. As provided for herein, it can also be used to identify a center, an approximate center, or other desired location of the glenoid 1018. Sizer attachments 340 of various lengths and having variously sized plate portions 342b of various diameters (e.g., extra small, small, medium, large, extra large) can be provided with the guide 300 to accommodate varying patient humeral resection surface diameters and anatomies. The illustrated embodiment of the attachment 340 has a medium diameter, as designated by the “M” shown in FIG. 16A. The sizes of the sizer attachments 340 can be based, for example, on a size of an implant (e.g., a stemless implant) and/or a reamer or reamer attachment that will be used to ream the surface 1015.

In at least some instances, an appropriate sized sizer attachment 340 can be chosen such that an outer edge of the plate portion 342b lies within the cortex border of the humeral resection surface 1015. Often it can be helpful for a perimeter of the circular body 341 to lie immediately within the cortex border and not contact any portion of the cortex perimeter. As shown in FIG. 16B, markings or indicators 345 on the arm portion 342a of the sizer attachment 340 can allow the sizer attachment 340 to center and measure the humeral resection surface 1015 with a circumference larger than the circular body 341. The markings 345 can be laser lines or other types of markings known to those skilled in the art. The circular body 341 can be substantially flat in shape and designed to enter a rotator interval 1020 above the subscapularis.

Once in the joint space, the plate portion 342b can be aligned parallel, or substantially parallel, and against the humeral resection surface 1015 such that the opening 343a, 343b mark the center point, or the approximate center point, of the humeral resection surface 1015. The alignment of the center opening 343a with the center point of the humeral resection surface 1015 can place the drill cannula 350 at the correct location on the lateral cortex 1023 of the humerus 1012 such that when a drill bit or guide pin 380 is passed through the drill cannula 350 and humerus 1012, a distal tip 382 disposed at a distal end 380d of the drill 380 exits center and perpendicular, or substantially perpendicular, also referred to as orthogonal or substantially orthogonal, to the humeral resection surface 1015. The distal tip 382 can be used to puncture tissue and/or drill in bone as desired. The humeral sizer attachment 340 further operates with the distal tip 350t of the drill cannula 350 to create a clamping force on the humeral resection surface 1015, which can maintain the position of the guide on the humerus until bone pins 370a, 370b are passed through the fixation feature(s) 330a, 330b, as shown through the bone pin clamps 332a, 332b, to secure the guide 300 to the bone.

A person skilled in the art will appreciate sizer plates, such as INHANCE™ humeral head trials (from Johnson & Johnson of New Brunswick, NJ), can also be used in conjunction with, or in lieu of, the humeral sizer attachment 340 and/or other similarly designed humeral sizer attachments. By way of example, FIG. 16D illustrates one example of a sizer plate 1172 that can be attached to a humeral sizer attachment like the humeral sizer attachment 340 for use in the same. The sizer plate 1172 can be manufactured in various diameters to accommodate varying patient humeral resection surface diameters. In at least some instances, an appropriate sized sizer plate 1172 can be chosen such that an outer edge of the sizer plate 1172 lies within the cortex border of the humeral resection surface 1015. Often it can be helpful for a perimeter of the sizer plate 1172 to lie immediately within the cortex border and not contact any portion of the cortex perimeter. Both the circular body 341 and the sizer plate 1172 can be substantially flat in shape and designed to enter a rotator interval 1020 above the subscapularis. The sizer plate 1172 can be considered to be a twist-on sizer because it is configured to be twisted onto and off a sizer attachment similar to the sizer attachment 340. The plate 1172 can be coupled to a sizer attachment like the sizer attachment 340 prior to or after the sizer attachment is coupled to the distal end 310d of the arm 310.

As shown in FIG. 16D, a circular recess 1174 can be formed in a lower surface of the sizer plate 1172, the lower surface being the surface that faces the attachment 340. The recess 1174 can be sized to seat on the attachment 340 such that the circular body 341 of the rigid base 342 sits within it. One or more mating features, non-limiting examples of which include a tang 1175 and a plurality of tabs 1176, can be used to help mate the sizer plate 1172 to a sizer attachment like the attachment 340 and a central opening 1178 can be formed to assist in locating a center, or an approximate center, of a location on bone. An arm 1179 can be part of a proximal portion 1172p of the plate 1172, with the arm including opposed engagement tabs 1179t, to further assist in mating the plate 1172 to an attachment like the attachment 340.

### Use of Humeral Guide to Center Mark Glenoid

The humeral guide 300 with the sizer attachment 340 coupled thereto can be used in conjunction with identifying a center or approximate center of a glenoid, and forming a starter hole or pilot hole in the glenoid. The guide 300 can be fixed to the humerus as shown in FIG. 15 by introducing the sizer attachment 340 to the surgical site through the rotator interval 1020, above the subscapularis, and an arm portion of the rigid base 342, and thus the arm 1179 of the sizer plate 1172, can be aligned with a bicipital groove 1011 that separates a greater tubercle from a lesser tubercle of the humerus 1012. This can provide optimal access and visualization through the rotator interval 1020 during the course of the procedure. One or more retractors can be placed between a rim of the glenoid 1018 and the humeral cut surface 1015 to maximize visualization of joint space. In at least some instances it may be advantageous to extend an incision being used to insert instruments and the like to the surgical site to accommodate the drill cannula 350 contacting bone.

The sizer attachment 340 can be placed planar with the humeral resection surface 1015 and retractors and/or the tang 1175 can be used to locate the cortical rim to help centralize the sizer plate 1172. More particularly, the sizer plate 1172 can be aligned with the humeral resection surface 1015 such that the perimeter of the sizer plate 1172 lies within the outer cortex layer of the humeral resection surface 1015. The tang 1175 of the sizer plate 1172 can grasp an edge of the humeral resection surface 1015, i.e., the cortical rim, to assist in centering the attachment 340 and sizer plate 1172 with respect to the humeral resection surface 1015. The central opening 1178 of the sizer plate 1172 can be aligned with the central opening 343a of the sizer attachment 340 to locate a center of the humeral resection surface 1015. Various sizer plates can be twisted onto and off the sizer attachment 340 to determine the appropriate size for subsequent attachments and/or the prosthesis to be used. In alternative embodiments, sizer plates are not used, and instead the attachment 340 is centered as described above with respect to FIGS. 16B-16C.

Once the sizer plate 1172 is centered on the humeral resection surface 1015, the drill cannula 350 can be pushed towards the humeral resection surface 1015 along the longitudinal axis LC, which is colinear with the longitudinal axis T extending through the transhumeral tunnel formed in the humerus 1012, as shown in FIG. 15, to engage the lateral cortex 1023 of the humerus 1012. As a result, the central opening 1178 of the sizer plate 1172, the central opening 343a of the sizer attachment 340, the hub opening 322, and the opening 356 of the drill cannula 350 can all be aligned along the longitudinal axes LC and T. The drill cannula 350 can, in turn, be locked in place. It is possible that a skin incision may need to be extended distally to accommodate the drill cannula 350 contacting the lateral cortex 1023. Aspects or features of the bone pin clamps 332a, 332b, and associated pins 370a, 370b can be operated to provide further adjustments for positioning the guide 300 with respect to the humerus 1012 to assist in placing the guide 300 at the desired location such that the sizer plate 1172 is centered on the humeral resection surface 1015 and the guide 300 is properly situated for performing the desired surgical procedure. The use of these features, and thus movement of the same (e.g., movement of the bone pin clamps 332a, 332b and associated bone pins 370a, 370b, as well as rods 336a, 336b supporting the clamps 332a, 332b) can occur before and/or after the drill cannula 350 is engaged with the lateral cortex 1023. By way of non-limiting examples, the bone pin clamps 332a, 332b can be loosened into an unlocked configuration at any time to make adjustments to the guide position before or after advancing the drill cannula 350 through the hub opening 322 and securing a position of the drill cannula 350 with respect to the hub 320.

After guide 300 has been placed at a desired location and position, the bone pins 370a, 370b can be introduced through the pin the bone pin clamps 332a, 332b to maintain the location and position of the guide 300 with respect to the humeral resection surface 1015, as shown in FIG. 15. The freedom of movement of the bone pins 370a, 370b within the bone pin clamps 332a, 332b and the freedom of movement of the bone pin clamps 332a, 332b on the associated support rods 336a, 336b allow the surgeon to guide the bone pins 370a, 370b into the humerus 1012 at various locations and angles with respect to the humerus 1012 and/or the guide 300.

The threads that are on the distal portion of the pins 370a, 370b can allow for measured insertion of the pins 370a, 370b into the humerus 1012, for example by rotating the pins 370a, 370b relative to the humerus 1012. If adequate bone pin purchase is not obtained based on the location of the bone pin clamps 332a, 332b, a readjustment of the bone pin clamps 332a, 332b location and/or position along the support rods 336a, 336b and/or a readjustment of the humeral guide 300 location and/or position may be appropriate. For example, after bone pins 370a, 370b are passed through the bone pin clamps 332a, 332b and into the humerus 1012, further micro adjustments can be made to the guide 300 position by unlocking the bone pin clamps 332a, 332b and/or shifting the guide 300 before locking the bone pin clamps 332a, 332b to secure the guide 300 with respect to the humerus 1012 and the surgical site more generally.

After the guide 300 is situated at its desired location to allow for actions like reaming the humerus 1012 and/or the glenoid 1018, the sizer plate 1172 can be detached from the sizer attachment 340. As illustrated in FIG. 16E, this can be accomplished, for example, by detaching the engagement tabs 1179t from the attachment body or base 342, and then removing the sizer plate 1172 from the joint space. More particularly, the engagement tabs 1179t can be pushed and the sizer plate 1172 disconnected from the attachment 340, for example by moving the portion that include the tabs 1179t in the direction R and then rotating the sizer plate 1172 to detach it from the attachment 340. Additional sizer plates 1172 can be attached and detached until centering of the guide 300 is accomplished and the proper size is determined for a reamer, prosthesis, and/or other equipment to be used and/or implanted at the surgical site. The guide 300 can remain at the set location for a remainder of the surgical procedure to assist in guiding other instruments for accurate glenoid and/or humeral bone preparation. The attachment 340 can likewise remain attached to the guide 300 for at least some portion of procedures that are subsequently performed.

In some embodiments, the centering and sizing can be further confirmed by introducing another sizer component, as shown an INHANCE humeral sizer or sizer plate 1172′ available from DePuy Synthes, as shown in FIG. 16F. The INHANCE humeral sizer 1172′ can include a central boss (not visible) that extends distally in the illustrated embodiment of FIG. 16F, towards the attachment 340. A central opening 178′ can extend through the sizer 1172′, including through the central boss. The boss can be inserted into the opening 343a of the attachment 340 such that the opening 343a and the central opening 1178′ are aligned. As shown, the sizer 1172′ can sit immediately inside the cortical rim and at no point contacts the cortical rim. During a procedure, a user can evaluate this positioning of the sizer 1172′ with respect to the humeral resection surface 1015. After the positioning of the guide 300 has been confirmed, a user can ensure that all connection points are fully secured, including but not limited to the bone pins 370a, 370b, the rods 336a, 336b, the bone pin clamps 332a, 332b, the drill cannula 350, and the attachment 340.

A drill bit 380′ can be passed into the opening 356 of the drill cannula 350 by advancing it towards the humeral resection surface 1015. The drill bit 380′ can be, for example, a pilot drill, such as a 4.0 mm drill, or any of the other drills disclosed herein or known to those skilled in the art. The drill bit 180′ can pass into and through the drill cannula 350, into and through the humerus 1012, and into and through the central opening 343a in the sizer attachment 340, with a tip 382′ of the drill bit 380′ extending into the joint space, substantially centered on and substantially perpendicular to the humeral resection surface 1015, as shown in FIG. 17. The drill bit 380′ can be operated, for example by applying power to a hub, which can be a modified trinkle connection, to advance and/or rotate the drill bit 380′.

Once this positioning of the drill bit 180′ proximate to the humeral resection surface 1015 has been confirmed, the drill bit 180′ can be slightly retracted such that the drill bit 380′ is below the plane of the humeral resection surface 1015 and the drill bit 380′ can be detached from power. The sizer attachment 340 may be removed to provide increased visualization of the surgical site. As shown in FIG. 17, a knob 348 of the attachment 340 can be rotated in a counterclockwise direction to allow the sizer attachment 340 to be disconnected from the distal end 310d of the arm 310 and removed from the surgical site, for instance by moving it through the rotator interval 1020, as illustrated by an arrow W. After the sizer attachment 340 is disconnected, the tension supplied by the associated drill cannula, e.g. the drill cannula 350 (not shown) to the humerus 1012 may need to be adjusted to ensure rigidity of the humeral guide 300 before performing subsequent tasks, like humeral reaming.

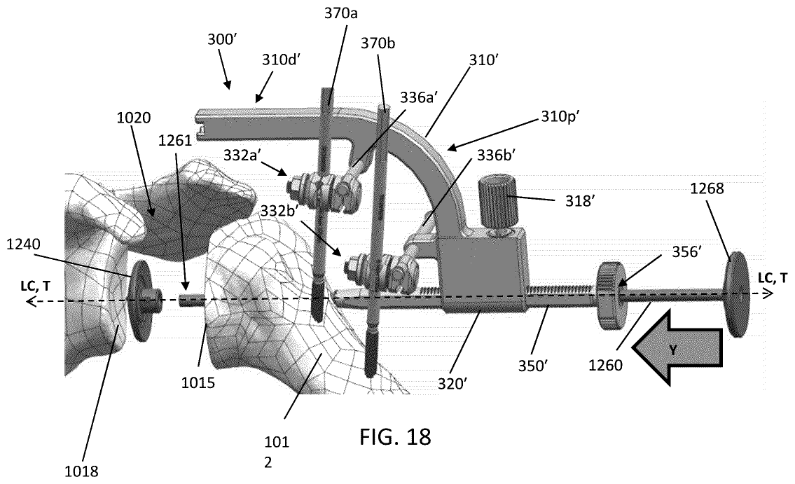

With the humeral resection surface 1015 center identified, now actions can be performed to use the guide 300 to identify a center, or approximate center, of the glenoid 1018. In some embodiments, it can be helpful to position the arm in adduction, for example by providing a slight external rotation, to perform this task. This can be achieved, for example, by rotating the humerus 1012 in the direction R, as shown in FIG. 2B. Positioning the arm in this manner can aid in the assembly of components used in conjunction with the guide 300. The resulting configuration is illustrated in FIG. 18, although a different humeral guide 300′ is illustrated.

The purpose of FIG. 18 is to illustrate what the configuration looks like when a humeral guide, as shown the guide 300′, is coupled to the humerus 1012 and used to perform the center identification and/or reaming actions described above at least with respect to FIGS. 7A-10E. Accordingly, a further description of how the center identification and reaming actions are performed is unnecessary. Although the guide 300′ has some different features than the guide 300, many of the core components of the two embodiments are similar and/or at least serve a similar purpose. A person skilled in the art will appreciate how the guide 300 would look in a similar set-up as shown in FIG. 18.

As shown in FIG. 18, the guide 300′ includes an arm 310′ having proximal and distal portions 310p′, 310d′ and a carriage or hub 320′. A cannulated bullet or drill cannula 350′ can be disposed within the hub 320′, the drill cannula 350′ being able to receive a guide, like the cannulated drill guide 1260, among other similar objects described herein, including but not limited to the drill bit 285, the driver 290, the drill bits 380, 380′, the guidewire 7000, and the drill bit 1285. Further, support rods 336a′, 336b′, bone pin clamps 332a′, 332b′, and bone pins 370a, 370b can be provided and operated in similar manners as provided for herein and otherwise understood by those skilled in the art in view of the present disclosures. In the illustrated embodiment, the sizer 1240 is illustrated as the component disposed in the glenohumeral joint space, for instance by introducing it through the rotator interval 1020, and the distal end 1261 of the cannulated drill guide 1260 can be moved in a direction Y by pushing and/or applying a slight twisting motion on the proximal end 1268 of the cannulated drill guide 1260 to eventually mate the sizer 1240 to the cannulated drill guide 1260. This is akin to operation of the drill guide 1260 and the sizer 1240 in FIG. 9E, with now the humeral guide 300′ being operated to assist in identifying the center, or approximate center, and sizing the glenoid 1018. Similarly, the other actions illustrated herein, such as the actions illustrated using the glenoid guide 200 with respect to FIGS. 7A-8B, can also be performed using the guide 300, the guide 300′, and other humeral guides provided for herein or otherwise derivable from the present disclosures. Likewise, the actions illustrated in FIGS. 10A-10E can also be performed using humeral guides (e.g., the guides 300, 300′), or glenoid guides (e.g., the guides 200, 200′), although that may not be preferred in at least some instances at least because of the impact the use of such guides may have on visualization of the surgical site.

### Other Considerations

The devices, tools, components, and the like described herein can be processed before use in a surgical procedure. First, a new or used instrument can be obtained and, if necessary, cleaned. The instrument can then be sterilized. In one sterilization technique, the instrument can be placed in a closed and sealed container, such as a plastic or TYVEK bag. The container and its contents can then be placed in a field of radiation that can penetrate the container, such as gamma radiation, x-rays, or high-energy electrons. The radiation can kill bacteria on the instrument and in the container. The sterilized instrument can then be stored in the sterile container. The sealed container can keep the instrument sterile until it is opened in the medical facility. Other forms of sterilization known in the art are also possible. This can include beta or other forms of radiation, ethylene oxide, steam, or a liquid bath (e.g., cold soak). Certain forms of sterilization may be better suited to use with different portions of the devices, tools, components, and the like due to the materials utilized, the presence of electrical components, etc.

Examples of the above-described embodiments can include the following:
1. A method for reaming a glenoid, comprising:
   disposing a reamer attachment in a glenohumeral joint space, the glenohumeral joint space being located between a humerus and a glenoid;
   passing a driver through a transhumeral tunnel formed in the humerus such that a distal end of the driver extends into the glenohumeral joint space;
   coupling the reamer attachment to the distal end of the driver; and
   operating the driver to rotate the reamer attachment and advance the reamer attachment into a surface of the glenoid.
2. The method of example 1, further comprising:
   prior to disposing the reamer attachment in the glenohumeral joint space, coupling a glenoid guide to the humerus such that a drill cannula associated with the glenoid guide is in contact with the humerus;
   passing a drill through the drill cannula to form the transhumeral tunnel.
3. The method of example 2, further comprising:
   operating a drill to form a pilot hole in a surface of the glenoid;
   docking a tip of the drill in the pilot hole;
   adjusting a location of the glenoid guide with respect to the humerus to set at least one of a version or inclination for the glenoid guide.
4. The method of example 3, further comprising:
   disposing a glenoid sizer having an open slot formed therein in the glenohumeral joint space;
   passing the glenoid sizer onto the drill, the drill passing through the slot and to a central opening of the glenoid sizer; and
   using the glenoid sizer to at least one of identify an approximate center of the surface of the glenoid, determine a size of the glenoid, or identify a trajectory for using the glenoid guide to ream the surface of the glenoid.
5. The method of any of examples 2 to 4, wherein the glenoid guide comprises an offset arm having a proximal opening formed in a proximal portion of the offset arm, a distal opening formed in a distal portion of the offset arm, the proximal and distal openings being colinear, and an intermediate portion that extends offset from a longitudinal axis extending between the proximal and distal openings such that the longitudinal axis can extend through a humerus when coupled thereto while the intermediate portion can extend around the humerus.
6. The method of example 1, further comprising:
   prior to disposing the reamer attachment in the glenohumeral joint space, coupling a humeral guide to the humerus such that a drill cannula associated with the humeral guide is in contact with the humerus and a humeral sizer attachment coupled to the humeral guide is in contact with a humeral resection surface; and
   passing a drill through the drill cannula to form the transhumeral tunnel.
7. The method of example 6, further comprising locating an approximate center of the humeral resection surface, the approximate center being located at a terminal end of the transhumeral tunnel.
8. The method of example 6 or 7, coupling a humeral guide to the humerus comprises inserting at least one bone pin into the humerus, the bone pin being coupled to a bone pin clamp that is coupled to the humeral guide.
9. The method of example 8, further comprising adjusting the bone pin clamp in at least one degree of freedom to position the at least one bone pin at a desired location to be inserted into the humerus.
10. The method of example 9, further comprising adjusting the clamp in at least two degrees of freedom to position the at least one bone pin at a desired location to be inserted into the humerus.
11. The method of any of examples 7 to 10, wherein locating an approximate center of a humeral resection surface comprises:
   coupling a proximal end of the humeral sizer attachment to a distal end of an arm of the humeral guide;
   positioning a distal end of the humeral sizer attachment at a location proximate to the humeral resection surface, the distal end of the sizer attachment including a plate having a central opening formed therein; and
   positioning the central opening of the plate of the distal end of the humeral sizer attachment such that it aligns with a longitudinal axis extending through the drill cannula and the transhumeral tunnel,
   wherein the drill cannula is moveably coupled to a proximal end of the arm of the humeral guide.
12. The method of any of examples 1 to 11, further comprising:
   prior to disposing the reamer attachment in the glenohumeral joint space, disposing a glenoid sizer in the glenohumeral joint space;
   passing a shaft through the transhumeral tunnel formed in the humerus such that a distal end of the shaft extends into the glenohumeral joint space;
   coupling the glenoid sizer to the distal end of the shaft; and
   operating the shaft to cause the glenoid sizer to at least one of be proximate to or in contact with the surface of the glenoid in conjunction with at least one of identifying an approximate center of the surface of the glenoid or determining a size of the glenoid.
13. The method of example 12, wherein disposing a glenoid sizer in the glenohumeral joint space further comprises passing a sizer handle having the glenoid sizer coupled to a distal end thereof through a rotator interval to dispose the glenoid sizer in the glenohumeral joint space.
14. The method of any of examples 1 to 13, further comprising prior to disposing the reamer attachment in the glenohumeral joint space, passing a drill bit into and through the glenohumeral joint space and into a surface of the glenoid to form at least one of a starter hole or a pilot hole, the drill bit not passing through the humerus.
15. The method of any of examples 1 to 13, further comprising prior to disposing the reamer attachment in the glenohumeral joint space, passing a drill bit through the transhumeral tunnel formed in the humerus, through the glenohumeral joint space, and into a surface of the glenoid to form at least one of a starter hole or a pilot hole.
16. The method of example 14 or 15, wherein the at least one of a starter hole or a pilot hole is formed at an identified approximate center of the surface of the glenoid.
17. The method of any of examples 2, 3, 4, 14, 15, or 16, wherein the drill bit is a stepped drill bit having a distal portion with a smaller diameter than a portion of the drill bit that is proximal of the distal portion.
18. The method of any of examples 1 to 17, further comprising:
   disposing a guidewire in the surface of the glenoid; and
   disposing the driver over the guidewire,
   wherein operating the driver to rotate the reamer attachment and advance the reamer attachment into a surface of the glenoid occurs while the guidewire is disposed within the driver and the reamer attachment is disposed radially around the guidewire.
19. The method of any of examples 1 to 17, further comprising engaging a distal tip of the driver with a pilot hole formed in an approximate center of the surface of the glenoid prior to operating the driver to rotate the reamer attachment and advance the reamer attachment into the surface of the glenoid.
20. The method of example 19, wherein the method is performed without the assistance of a guidewire disposed in the surface of the glenoid.
21. The method of any of examples 1 to 20, further comprising inserting an implant into the surface of the glenoid after the reamer attachment has treated the surface of the glenoid.
22. The method of example 21, wherein inserting an implant into the surface of the glenoid further comprises:
   decoupling the reamer attachment from the driver;
   removing the reamer attachment and the driver from the glenohumeral joint space;
   passing a distal end of the glenoid guide into the glenohumeral joint space, the distal end of the glenoid guide having coupled thereto the implant; and
   applying a force to a proximal end of the glenoid guide to dispose the implant in the surface of the glenoid.
23. The method of example 22, further comprising:
   decoupling the implant from distal end of the glenoid guide;
   removing the glenoid guide from the glenohumeral joint space;
   passing the distal end of the glenoid guide into the glenohumeral joint space, the distal end of the glenoid guide having coupled thereto an impactor; and
   applying a force to the proximal end of the glenoid guide to use the impactor to further dispose the implant in the surface of the glenoid.
24. The method of any of examples 21 to 24, further comprising:
   prior to inserting an implant into the surface of the glenoid and after the reamer attachment has treated the surface of the glenoid, passing a distal end of the glenoid guide into the glenohumeral joint space, the distal end of the glenoid guide having coupled thereto an implant trial;
   applying a force to a proximal end of the glenoid guide to dispose the implant trial in the surface of the glenoid; and
   using the implant trial to determine at least one of an appropriate configuration of the implant, a size of the implant, or a location to dispose the implant in the surface of the glenoid.
25. The method of any of examples 21 to 24, wherein the glenoid guide comprises an offset arm having a proximal opening formed in a proximal portion of the offset arm and a distal opening formed in a distal portion of the offset arm are colinear and an intermediate portion of the arm extends offset from a longitudinal axis extending between the distal opening and the proximal opening, such that the longitudinal axis can extend through a humerus when coupled thereto while the intermediate portion can extend around the humerus.
26. The method of any of examples 1 to 25, wherein a subscapularis tendon is intact during an entirety of the method.
27. The method of example 26, wherein the reamer attachment is inserted to the glenohumeral joint space at least one of superior to the intact subscapularis tendon or inferior to the intact subscapularis tendon.
28. The method of example 26 or 27, further comprising manipulating the subscapularis tendon to increase visibility by moving it away from its natural location while keeping it intact.
29. The method of any of examples 1 to 28, wherein the method is performed while the humerus is not distracted from its associated joint.
30. The method of any of examples 1 to 29, wherein the method is performed while the humerus is in an adducted position.
31. A system for reaming a glenoid, comprising:
   a reamer attachment having a central opening formed therethrough and one or more cutting surfaces disposed radially outward from the central opening; and
   a driver configured to pass into the central opening of the reamer attachment to selectively couple and de-couple from the reamer attachment within a glenohumeral joint space such that the reamer attachment and the driver can be disposed within the glenohumeral joint space separate from each other.
32. The system of example 31, wherein the one or more cutting surfaces disposed radially outward from the central opening comprise:
   a first cutting surface disposed at a distal end of the reamer attachment; and
   a second cutting surface disposed proximal of the first cutting surface, and disposed further radially outward from the central opening than the first cutting surface.
33. The system of example 31 or 32,
   wherein an inner wall of the reamer attachment comprises a keyed portion, and
   wherein the driver comprises a keyed portion that is complementary to the keyed portion of the reamer attachment such that when the keyed portion of the reamer attachment is mated with the keyed portion of the driver, the reamer attachment and driver are coupled together in a manner that allows the driver to rotate and translate the reamer attachment.
34. The system of any of examples 31 to 33, wherein a distal tip of the driver comprises a bullet-shaped tip.
35. The system of any of examples 31 to 34, further comprising a glenoid guide having an offset arm having a proximal opening formed in a proximal portion of the offset arm and a distal opening formed in a distal portion of the offset arm are colinear and an intermediate portion of the arm extends offset from a longitudinal axis extending between the distal opening and the proximal opening such that the longitudinal axis can extend through a humerus when coupled thereto while the intermediate portion can extend around the humerus.
36. The system of example 35, the glenoid guide further comprising a drill cannula configured to be coupled to the proximal portion of the arm, the drill cannula being able to pass through and be selectively held within the proximal opening by the glenoid guide.
37. The system of example 35 or 36, the glenoid guide further comprising a post disposed as part of the distal portion, the post being configured to receive an implant thereon.
38. The system of any of examples 31 to 37, further comprising a glenoid sizer having a central opening formed therein, the glenoid sizer being configured to mate to at least one of the driver or another shaft, and the glenoid sizer being configured to assist in at least one of identifying an approximate center of the glenoid or determining a size or one or more implants to be used in conjunction with the glenoid.
39. A glenoid guide, comprising:
   a proximal portion having a proximal opening formed therein;
   a distal portion having a distal opening formed therein, the distal opening being colinear with the proximal opening; and
   an offset arm extending from the proximal portion to the distal portion, the offset arm being offset with respect to a longitudinal axis extending through the distal and proximal openings,
   wherein the glenoid guide is configured such that the longitudinal axis can extend through a humerus when coupled thereto while the offset arm extends around the humerus.
40. The glenoid guide of example 39, further comprising a drill cannula configured to be coupled to the proximal portion, the drill cannula being able to pass through and be selectively held within the proximal opening by the glenoid guide.
41. The system of example 39 or 40, the glenoid guide further comprising a post disposed as part of the distal portion, the post being configured to receive an implant thereon.

Although the procedures provided for herein are described in conjunction with performing an anatomical shoulder procedure in which the resected humeral head is replaced with a prosthesis that mimics a humeral head, the instruments and procedures provided for herein can also be used and applied in a reverse shoulder procedure in which the humeral head prosthesis is disposed on the glenoid and the implant placed on the humerus is a humeral head prosthesis receiving surface. A person skilled in the art, in view of the present disclosures, will understand how reverse shoulder techniques can be implemented in view of the present disclosures.

One skilled in the art will appreciate further features and advantages of the disclosure based on the above-described embodiments and techniques. Accordingly, the disclosure is not to be limited by what has been particularly shown and described, except as indicated by the appended claims. By way of example, while the present disclosure primarily focuses on shoulder arthroplasty procedures and the use of guides and other components, instruments, tools, implants, etc. in conjunction with the same, the disclosed techniques, devices, and the like can be used and/or adapted for use with other shoulder procedures and/or for use in surgical procedures in other locations in the body. Accordingly, references to shoulder anatomy and/or guides herein being “humeral” or for the “glenoid” are not limiting to such use, and the disclosures herein can be used in procedures and guides for other anatomies (e.g., boney anatomies), whether human or other animals. A person skilled in the art, in view of the present disclosures, will be able to adapt some or all of the various systems, instruments, tools, and techniques disclosed herein for use in surgical procedures in other locations and/or for use with non-humans.

Further, a person skilled in the art will appreciate that various features or other disclosures associated with one embodiment of a device, system, component, and/or surgical technique can be used in other devices, systems, components, and/or surgical techniques disclosed herein or otherwise derivable therefrom. It is within the skill of a person skilled in the art to be able to apply teachings, or part of teachings, from one such device, system, component, and/or surgical technique to one or more other devices, systems, components, and/or surgical techniques. To the extent the present disclosure does not describe materials that can be used to manufacture the guides and other components, instruments, tools, implants, etc. disclosed herein and/or does not identify particular dimensions and the like for the guides and other components, instruments, tools, implants, etc., a person skilled in the art will appreciate typical materials and dimensions that are appropriate. All publications and references cited herein are expressly incorporated herein by reference in their entirety. Further, U.S. Provisional Patent Application No. 63/689,631, entitled “Smart Shoulder Tissue Sparing Approach Techniques and Related Instrumentation,” is incorporated by reference herein in its entirety.

What is claimed is:

1. A method for reaming a glenoid, comprising:
disposing a reamer attachment in a glenohumeral joint space, the glenohumeral joint space being located between a humerus and a glenoid;
passing a driver through a transhumeral tunnel formed in the humerus such that a distal end of the driver extends into the glenohumeral joint space;
coupling the reamer attachment to the distal end of the driver; and
operating the driver to rotate the reamer attachment and advance the reamer attachment into a surface of the glenoid.

2. The method of claim 1, further comprising:
prior to disposing the reamer attachment in the glenohumeral joint space, coupling a glenoid guide to the humerus such that a drill cannula associated with the glenoid guide is in contact with the humerus;
passing a drill through the drill cannula to form the transhumeral tunnel.

3. The method of claim 2, further comprising:
operating a drill to form a pilot hole in a surface of the glenoid;
docking a tip of the drill in the pilot hole;
adjusting a location of the glenoid guide with respect to the humerus to set at least one of a version or inclination for the glenoid guide.

4. The method of claim 3, further comprising:
disposing a glenoid sizer having an open slot formed therein in the glenohumeral joint space;
passing the glenoid sizer onto the drill, the drill passing through the slot and to a central opening of the glenoid sizer; and
using the glenoid sizer to at least one of identify an approximate center of the surface of the glenoid, determine a size of the glenoid, or identify a trajectory for using the glenoid guide to ream the surface of the glenoid.

5. The method of claim 2, wherein the glenoid guide comprises an offset arm having a proximal opening formed in a proximal portion of the offset arm, a distal opening formed in a distal portion of the offset arm, the proximal and distal openings being colinear, and an intermediate portion that extends offset from a longitudinal axis extending between the proximal and distal openings such that the longitudinal axis can extend through a humerus when coupled thereto while the intermediate portion can extend around the humerus.

6. The method of claim 2, wherein the drill bit is a stepped drill bit having a distal portion with a smaller diameter than a portion of the drill bit that is proximal of the distal portion.

7. The method of claim 1, further comprising:
prior to disposing the reamer attachment in the glenohumeral joint space, disposing a glenoid sizer in the glenohumeral joint space;
passing a shaft through the transhumeral tunnel formed in the humerus such that a distal end of the shaft extends into the glenohumeral joint space;
coupling the glenoid sizer to the distal end of the shaft; and
operating the shaft to cause the glenoid sizer to at least one of be proximate to or in contact with the surface of the glenoid in conjunction with at least one of identifying an approximate center of the surface of the glenoid or determining a size of the glenoid.

8. The method of claim 1, further comprising prior to disposing the reamer attachment in the glenohumeral joint space, passing a drill bit into and through the glenohumeral joint space and into a surface of the glenoid to form at least one of a starter hole or a pilot hole, the drill bit not passing through the humerus.

9. The method of claim 1, further comprising inserting an implant into the surface of the glenoid after the reamer attachment has treated the surface of the glenoid.

10. The method of claim 9, wherein inserting an implant into the surface of the glenoid further comprises:
decoupling the reamer attachment from the driver;
removing the reamer attachment and the driver from the glenohumeral joint space;
passing a distal end of the glenoid guide into the glenohumeral joint space, the distal end of the glenoid guide having coupled thereto the implant; and
applying a force to a proximal end of the glenoid guide to dispose the implant in the surface of the glenoid.

11. The method of claim 9, wherein the glenoid guide comprises an offset arm having a proximal opening formed in a proximal portion of the offset arm and a distal opening formed in a distal portion of the offset arm are colinear and an intermediate portion of the arm extends offset from a longitudinal axis extending between the distal opening and the proximal opening, such that the longitudinal axis can extend through a humerus when coupled thereto while the intermediate portion can extend around the humerus.

12. The method of claim 1, wherein a subscapularis tendon is intact during an entirety of the method.

13. The method of claim 12, wherein the reamer attachment is inserted to the glenohumeral joint space at least one of superior to the intact subscapularis tendon or inferior to the intact subscapularis tendon.

14. The method of claim 1 wherein the method is performed while the humerus is not distracted from its associated joint.

15. The method of claim 1, wherein the method is performed while the humerus is in an adducted position.

16. A system for reaming a glenoid, comprising:
a reamer attachment having a central opening formed therethrough and one or more cutting surfaces disposed radially outward from the central opening; and
a driver configured to pass into the central opening of the reamer attachment to selectively couple and de-couple from the reamer attachment within a glenohumeral joint space such that the reamer attachment and the driver can be disposed within the glenohumeral joint space separate from each other.

17. The system of claim 16,
wherein an inner wall of the reamer attachment comprises a keyed portion, and
wherein the driver comprises a keyed portion that is complementary to the keyed portion of the reamer attachment such that when the keyed portion of the reamer attachment is mated with the keyed portion of the driver, the reamer attachment and driver are coupled together in a manner that allows the driver to rotate and translate the reamer attachment.

18. The system of claim 16, further comprising a glenoid guide having an offset arm having a proximal opening formed in a proximal portion of the offset arm and a distal opening formed in a distal portion of the offset arm are colinear and an intermediate portion of the arm extends offset from a longitudinal axis extending between the distal opening and the proximal opening such that the longitudinal axis can extend through a humerus when coupled thereto while the intermediate portion can extend around the humerus.

19. A glenoid guide, comprising:
a proximal portion having a proximal opening formed therein;
a distal portion having a distal opening formed therein, the distal opening being colinear with the proximal opening; and
an offset arm extending from the proximal portion to the distal portion, the offset arm being offset with respect to a longitudinal axis extending through the distal and proximal openings,
wherein the glenoid guide is configured such that the longitudinal axis can extend through a humerus when coupled thereto while the offset arm extends around the humerus.

20. The glenoid guide of claim 19, further comprising a drill cannula configured to be coupled to the proximal portion, the drill cannula being able to pass through and be selectively held within the proximal opening by the glenoid guide.
